(19) United States
(12) Patent Application Publication
(10) Pub. No.: US 2017/0261507 A1
(43) Pub. Date: Sep. 14, 2017

(54) Methods for Predicting and Monitoring Cancer Patients' Response to Teatment by Measuring Myeloid Derived Suppressor Cells (MDSCs)

(71) Applicants: YISSUM RESEARCH DEVELOPMENT COMPANY OF THE HEBREW UNIVERSITY OF JERUSALEM LTD., Jerusalem (IL); HADASIT MEDICAL RESEARCH SERVICE & DEVELOPMENT LIMITED, Jerusalem (IL)

(72) Inventors: Michal BANIYASH, Mevasseret Zion (IL); Michal LOTEM, Makabim-Re'ut (IL); Moshe SADE-FELDMAN, Modiin (IL); Julia KANTERMAN, Modiin (IL)

(21) Appl. No.: 15/505,318

(22) PCT Filed: Aug. 19, 2015
(86) PCT No.: PCT/IL2015/050837

§ 371 Date: Feb. 21, 2017

Related U.S. Application Data
(60) Provisional application No. 62/039,156, filed on Aug. 19, 2014.
(60) Provisional application No. 62/079,018, filed on Nov. 13, 2014.

(51) Int. Cl. G01N 33/569; C12Q 1/68; C07K 16/28; G01N 33/574; G01N 33/84

(52) U.S. Cl. CPC G01N 33/56972; G01N 33/5743; G01N 33/57419; G01N 33/84; C07K 16/2818; C12Q 1/6886; G01N 2333/70553; G01N 2333/70503; G01N 2333/70539; G01N 2800/52; G01N 2800/7028; C07K 2317/76; A61K 2039/505

(57) ABSTRACT

Provided herein are methods and kits for predicting and monitoring a cancer patient's response to treatment with a therapeutic agent by measuring the amount of myeloid derived suppressor cells (MDSC) having the profile CDl11b⁺CD33⁺HLA-DR⁻ and/or CDl11b⁺CD33⁺HLA-DR^{low}, and optionally by further measuring various suppressive features of the patient's immune system. Also provided herein are methods of treating a cancer patient comprising as an initial step determining whether the cancer patient would be responsive to treatment with the therapeutic agent as described above and wherein the patient is found to be responsive, administering the therapeutic agent.

### TECHNOLOGICAL FIELD

The invention is in the field of cancer therapy and in particular, the invention concerns methods for determining a cancer patient's therapy, as well as predicting and monitoring a cancer patient's response to therapy with a chemotherapeutic or an immunotherapeutic agent.

### BACKGROUND ART

References considered to be relevant as background to the presently disclosed subject matter are listed below:
WO 2012/0276004
WO 2012/149416
WO 2013/050998

Acknowledgement of the above references herein is not to be inferred as meaning that these are in any way relevant to the patentability of the presently disclosed subject matter.

### BACKGROUND

Many tumors are characterized by chronic inflammation-induced immunosuppression mediated by pro-inflammatory cells and mediators (1-4), which subvert the outcome of anti-cancer therapy. MDSCs are the main cell population causing immunosuppression in numerous cancers including CRC (3, 5-8). MDSCs are immature myeloid cells expanded in the course of chronic inflammation.

Such generated conditions manipulate the host's immune system; suppressing the innate and adaptive immune responses, as reflected by the impaired function of T and NK cells and is associated with down regulated expression of the CD247 and SNX9 molecules. Such chronic inflammation-induced immunosuppressive features act as critical barriers to effective anti-tumor responses and therapies.

WO 2012/0276004 discloses methods for determining the presence of cancer, monitoring cancer progression, cancer relapse, or cancer staging in a subject by evaluating specific MDSC cell populations.

WO 2012/149416 discloses a method of diagnosing, treating, or determining efficacy of treatment of a cancer patient, in particular a lymphoma patient, including a step of assessing the level of MDSC of specific phenotypes in the patient.

WO 2013/050998 discloses a method for determining the efficacy of treatment of a subject suffering from a chronic inflammatory condition comprising determining the level of expression of T cell antigen receptor (TCR) chain (CD247). Optionally, this method may further include determining the MDSC population in the subject.

Chemotherapeutic drugs commonly used to treat cancer affect not only the tumor but also the immune system, having a crucial impact on anti-tumor responses and disease outcome (5, 9).

### GENERAL DESCRIPTION

In a first of its aspects, the present invention provides a method for predicting a cancer patient's response to treatment with a therapeutic agent, said method comprising the steps of:
(a) Measuring the amount of myeloid derived suppressor cells (MDSC) having the profile CD11b⁺CD33⁺HLA-DR⁻ and/or CD11b⁺CD33⁺HLA-DR^{low}, in at least one biological sample obtained from the patient; and
(b) Determining whether the amount of MDSC of said profile is higher than a predetermined standard value or higher than a control sample;
   wherein
(i) said therapeutic agent is a chemotherapeutic agent or an immunotherapeutic agent or a combination thereof, and wherein
(ii) detection of a high amount of MDSC of the above profile in the at least one biological sample as compared with the predetermined standard value or the control sample indicates that the patient will not respond or will poorly respond to treatment with said agent.

In one embodiment, said method is for at least one of the following:
(a) Including or excluding patients in a clinical study;
(b) deciding whether the patient should start, continue or cease therapy with said chemotherapeutic or immunotherapeutic agent; or
(c) deciding which combination of chemotherapeutic and immunotherapeutic agents should be used.

In another aspect, the present invention provides a method for monitoring a cancer patient's response to treatment with a therapeutic agent, said method comprising the steps of:
(a) Measuring the amount of myeloid derived suppressor cells (MDSC) having the profile CD11b⁺CD33⁺HLA-DR⁻ and/or CD11b⁺CD33⁺HLA-DR^{low}, in at least one biological sample obtained from the patient; and
(b) Determining whether the amount of MDSC of said profile is higher than a predetermined standard value or higher than a control sample;
   wherein
(i) said therapeutic agent is a chemotherapeutic or immunotherapeutic agent or a combination thereof, and wherein
(ii) detection of a high amount of MDSC of the above profile in the at least one biological sample as compared with the predetermined standard value or the control sample indicates that the patient is not responding or is poorly responding to treatment.

In one embodiment, said method is used for determining the patient's therapeutic regime by at least one of the following:
(a) Discontinuing treatment with said chemotherapeutic agent, said immunotherapeutic agent or the combination thereof; or
(b) Combining the treatment with said chemotherapeutic agent, said immunotherapeutic agent or the combination thereof with at least one additional compound being an anti-inflammatory and/or an anti-MDSC therapeutic agent.

In one embodiment, said measuring of the amount of myeloid derived suppressor cells (MDSC) having the profile CD11b⁺CD33⁺HLA-DR⁻ and/or CD11b⁺CD33⁺HLA-DR^{low} is performed prior to treatment with said therapeutic agent.

In other embodiments, said measuring of the amount of myeloid derived suppressor cells (MDSC) having the profile CD11b⁺CD33⁺HLA-DR⁻ and/or CD11b⁺CD33⁺HLA-DR^{low} is performed at least once, preferably more than once, during treatment with said therapeutic agent.

In one embodiment said chemotherapeutic agent is selected from the group consisting of a DNA alkylating agent, a platinum compound, a Topoisomerase inhibitor, a Tyrosine kinase inhibitor, an antimetabolite drug, and any combination thereof.

In one specific embodiment, said chemotherapeutic agent is 5-fluoruracil (5FU) or Irinotecan (CPT11).

In another specific embodiment, said chemotherapeutic agent is a chemotherapy regimen selected from FOLFOX or FOLFIRI.

In one embodiment, said immunotherapeutic agent is selected from the group consisting of therapeutic antibodies, immune checkpoints inhibitors, cytokines, tumor infiltrating lymphocytes (TIL) and cancer vaccines.

In one specific embodiment, said immunotherapeutic agent is an anti CTLA4, anti PDL1 or anti PD1 agent.

In a specific embodiment said anti CTLA4 agent is the anti CTLA4 antibody Ipilimumab.

In another specific embodiment, said anti PD1 agent is the anti PD1 antibody lambrolizumab.

In certain embodiments, the cancer is selected from the group consisting of adrenal cancer, bladder cancer, brain cancer, breast cancer, cervical cancer, colorectal carcinoma, endometrial cancer, gastro-intestinal cancers, head and neck squamous cell carcinoma, leukemia, malignant lymphoma, including Hodgkin's lymphoma, liver cancer, melanoma, ovarian cancer, pancreatic cancer, prostate cancer, renal cancer, sarcoma, small cell lung cancer, non-small cell lung cancer, and thyroid cancer.

In one specific embodiment, the cancer is melanoma.

In one embodiment said melanoma is metastatic melanoma.

In another specific embodiment, the cancer is colorectal carcinoma.

In one embodiment, said colorectal carcinoma is metastatic colorectal carcinoma.

In certain embodiments said measuring of the amount of myeloid derived suppressor cells (MDSC) having the profile CD11b⁺CD33⁺HLA-DR⁻ and/or CD11b⁺CD33⁺HLA-DR^{low} is performed by a method comprising the step of contacting detecting molecules specific for MDSC with the biological sample.

In certain embodiments, said detecting molecules are labeled detecting molecules.

In other embodiments, said detecting molecules are attached to a substrate.

In certain embodiments, said detecting molecules are amino acid molecules or nucleic acid molecules.

In one specific embodiment, said amino acid molecules are antibodies that specifically recognize and bind MDSC having the profile CD11b⁺CD33⁺HLA-DR⁻ and/or CD11b⁺CD33⁺HLA-DR^{low}.

In certain embodiment said antibody-bound MDSC are detected using fluorescence activated cell sorter (FACS), immunohistology, ELISA, RIA or Western blotting.

In certain embodiments, said biological sample is any one of whole blood sample, fractionated blood sample, a spleen biopsy, cells obtained from lymph nodes, tissue biopsy or a tumor sample.

In certain embodiments said sample is a fresh sample, a preserved sample or a cryo-preserved sample.

In one embodiment the methods of the invention further comprise the step of determining the patient's metastatic severity prior to treatment with said therapeutic agent.

In certain embodiments, said method further comprises the step of at least one of:
(a) determining the expression levels of S100A8 and/or S100A9 proteins or encoding mRNA in said biological sample;
(b) determining the levels of cleaved caspase 3 in said biological sample;
(c) determining at least one of the level and/or activity of arginase 1 and iNOS in said biological sample;
(d) determining at least one of intracellular nitric oxide (NO) and reactive oxygen species (ROS) in said biological sample;
(e) determining the level of lactate dehydrogynase (LDH) in said biological sample;
(f) determining the level of MDSC suppressive activity on T cells in said biological sample.

In certain embodiments, said MDSC suppressive activity on T cells is measured by assessing down regulation of CD247 and/or SNX9 expression, and/or impaired T cell proliferation.

In one embodiment, determining elevated levels of NO, elevated levels of ROS, elevated levels of S100A8 and/or S100A9 proteins, low levels of cleaved caspase 3, and MDSC suppressive activity on T cells indicates that the patient will not respond to treatment with said chemotherapeutic agent, said immunotherapeutic agent or a combination thereof, or that the therapeutic regimen of said patient should be altered.

In another aspect, the present invention provides a method of treating a cancer patient with a therapeutic agent, said method comprising the steps of:
(a) Determining whether said patient is responsive to the therapeutic agent in accordance with the method described above; wherein said therapeutic agent is a chemotherapeutic agent or an immunotherapeutic agent or a combination thereof; and
(b) wherein the patient was determined to be responsive, administering to the patient an effective amount of said chemotherapeutic agent or said immunotherapeutic agent or a combination thereof.

In another aspect, the present invention provides a kit comprising:
(a) detecting molecules specific for MDSC having the profile CD11b⁺CD33⁺HLA-DR⁻ and/or CD11b⁺CD33⁺HLA-DR^{low}; and optionally further comprising at lease one of the following:
   i) At least one detecting molecule for determining LDH levels;
   ii) At least one detecting molecule specific for at least one of S100A8, S100A9, cleaved caspase 3, iNOS, arginase 1, NO, ROS, CD247 or SNX9;
   iii) secondary agents and/or buffers for performing detection of MDSC, LDH and at least one of S100A8, S100A9, cleaved caspase 3, iNOS, arginase 1, NO or ROS; and
(b) instructions for use.
   wherein said kit is for use in a method for predicting a cancer patient's response to treatment with a chemotherapeutic agent or an immunotherapeutic agent or a combination thereof, or for use in a method for determining whether the therapeutic regimen of a cancer patient should be altered.

In one embodiment, said altering the patient's therapeutic regimen comprises any one of:
(a) Refraining from treatment with said chemotherapeutic agent, said immunotherapeutic agent or the combination thereof;
(b) Discontinuing treatment with said chemotherapeutic agent, said immunotherapeutic agent or the combination thereof; or
(c) Combining the treatment with said chemotherapeutic agent, said immunotherapeutic agent or the combination thereof with at least one additional compound being an anti-inflammatory and/or an anti-MDSC therapeutic agent.

In one embodiment, said instructions for use comprise:
(a) Instructions for carrying out the measurement of the amount of MDSC; and optionally further comprise
(b) Instructions for carrying out the measurement of the amount of LDH levels; and optionally further comprise
(c) Instructions for carrying out the measurement of the amount of at least one of S100A8, S100A9, cleaved caspase 3, iNOS, arginase 1, NO, ROS, CD247 or SNX9; and
(d) Instructions for comparing the amount of MDSC and optionally of the LDH levels and optionally of the at least one of S100A8, S100A9, cleaved caspase 3, iNOS, arginase 1, NO, ROS, CD247 or SNX9 with a predetermined standard value or with a control sample.

In certain embodiments said detecting molecules are amino acid molecules or nucleic acid molecules.

In certain embodiments said detecting molecules are labeled detecting molecules.

In certain embodiments said detecting molecules are attached to a substrate.

In certain embodiments said amino acid molecules comprise antibodies that specifically recognize and bind MDSC.

In certain embodiments said secondary agents and/or buffers are suitable for performing fluorescence activated cell sorter (FACS) analysis, immunohistology, Western blotting, ELISA or RIA.

In another aspect, the present invention provides a method for selecting a melanoma patient suitable for receiving treatment with a therapeutic agent, said method comprising the steps of:
(a) Measuring the amount of myeloid derived suppressor cells (MDSC) having the profile CD11b⁺CD33⁺HLA-DR⁻ and/or CD11b⁺CD33⁺HLA-DR^{low}, in at least one biological sample obtained from the patient; and
(b) Determining whether the amount of MDSC is lower than a predetermined standard value or lower than a control sample;
wherein
   (i) said therapeutic agent is Ipilimumab or lambrolizumab or a combination thereof, and wherein
   (ii) detection of a low amount of MDSC in the at least one biological sample as compared with the predetermined standard value or the control sample, indicates that the patient is suitable for receiving treatment with Ipilimumab or lambrolizumab or a combination thereof.

In another aspect, the present invention provides a method for detecting changes in a cancer patient's disease development, said method comprising the steps of:
(a) Measuring the amount of myeloid derived suppressor cells (MDSC) having the profile CD11b⁺CD33⁺HLA-DR⁻ and/or CD11b⁺CD33⁺HLA-DR^{low}, in at least one biological sample obtained from the patient, at least one time point; and
(b) Determining whether the amount of MDSC of said profile is higher than a predetermined standard value or higher than a control sample;
   wherein
(i) said therapeutic agent is a chemotherapeutic or immunotherapeutic agent or a combination thereof, and wherein
(ii) detection of a high amount of MDSC of the above profile in the at least one biological sample as compared with the predetermined standard value or the control sample indicates that the patient's disease has progressed, and wherein detection of a low amount of MDSC of the above profile in the at least one biological sample as compared with the predetermined standard value or the control sample indicates that the patient's disease has regressed.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a schematic representation showing the percentage of CD33⁺CD11b⁺HLA-DR⁻ cells in peripheral blood obtained from healthy donors and from cancer patients (gated on HLA-DR^{-/-} cells). FIG. 1B is a schematic representation showing the CD247 expression presented as percent of mean fluorescence intensity (% MFI), in cells of healthy donors and cancer patients, gating was set on CD3⁺ cells. FIG. 1C is a schematic representation showing the correlation between MDSCs percentages and CD247 expression (shown as % MFI) in cancer patients; **P<0.0015; ***P<0.0001.

FIG. 2A is a schematic representation showing the percentage of CD33⁺CD11b⁺HLA-DR⁻ cells in the peripheral blood of two melanoma patient age groups: 30-60 years old and 60-90 years old. FIG. 2B is a schematic representation showing the percentage of CD33⁺CD11b⁺HLA-DR⁻ cells in the peripheral blood of two melanoma patient groups divided according to the tumor origin (skin or ocular). FIG. 2C is a schematic representation showing the percentage of CD33⁺CD11b⁺HLA-DR⁻ cells in the peripheral blood of two melanoma patient groups, one group received chemotherapeutic treatments (Chemo(+)) prior to ipilimumab treatments, and one group did not receive chemotherapeutic treatments (Chemo(−)) prior to ipilimumab treatments; n.s—not significant.

FIG. 3A is a schematic representation showing the percentage of CD33⁺CD11b⁺HLA-DR⁻ cells in the peripheral blood of two groups of melanoma patients: patients that responded to ipilimumab treatments (SD/CR; SD—stable disease, CR—complete response) and patients that did not respond to ipilimumab treatments (PD—progressive disease) (gated on HLA-DR^{-/-} cells). FIG. 3B is a schematic representation showing the lactate dehydrogenase (LDH) levels in the peripheral blood of patients that responded to ipilimumab treatments (SD/CR) and patients that did not respond to ipilimumab treatments (PD); **P<0.008, n.s—not significant.

FIG. 4A is a schematic representation showing the percentage of CD33⁺CD11b⁺HLA-DR⁻ cells in patients with metastatic severity defined as M1A, M1B, and M1C (M1A/B/C) and in patients with higher metastatic severity defined as M2. FIG. 4B is a schematic representation showing the percentage of CD33⁺CD11b⁺HLA-DR⁻ cells in patients with low LDH levels and with high LDH levels (LDH low<480 U/I; LDH high>480 U/I). FIG. 4C is a Kaplan-Meyer curve showing the percent survival in months of melanoma patients with Low level MDSC (<55.5%, n=39) and with High level MDSC (>55.5%, n=14). FIG. 4D is a schematic representation showing survival in months of patients in the Low level MDSC group (11.56±1.2, n=39) and the high level MDSC group (6.857±1.4, n=14); **P<0.008; *P<0.02.

FIG. 5A is a Kaplan-Meyer curve showing the percent survival in months of two groups of melanoma patients (I and II) divided according to their metastatic level prior to ipilimumab treatments; I-M1A/B/C; II-M2. FIG. 5B is a schematic representation showing survival in months of M1A/B/C group (12.4±1.2, n=35) and M2 group (6.1±1.1, n=18). FIG. 5C is a Kaplan-Meyer curve showing the percent survival in months of two groups of melanoma patients divided according to their LDH levels prior to ipilimumab treatments; LDH low<480 U/I; LDH high>480 U/I. FIG. 5D is a schematic representation showing survival in months of LDH low group (13.5±1.4; n=28) and LDH high group (6.6±0.9, n=20); ***P<0.0009; **P<0.0021.

FIG. 6 is a Kaplan-Meyer curve showing the percent survival in months of two groups of melanoma patients divided according to their initial parameters measured before the first ipilimumab treatment. One group had low MDSCs levels, low LDH levels and a M1A-C staging (MDSC↓LDH↓M1A-C); the second group had high MDSCs levels, high LDH levels and an M2 staging (MDSC↑LDH↑M2); P<0.0001.

FIG. 7A is a schematic representation showing the percentage of CD11b⁺CD33⁺HLA-DR⁻ MDSC in 23 CRC patients and in 20 healthy donors. FIG. 7B is a schematic representation showing NO⁻ levels produced by MDSCs from healthy donors and CRC-patients presented as MFI, gating on CD11b⁺CD33⁺HLA-DR⁻ cells. FIG. 7C is a schematic representation showing ROS levels produced by MDSCs from healthy donors and CRC-patients presented as MFI, gating on CD11b⁺CD33⁺HLA-DR⁻ cells. FIG. 7D is a schematic representation showing CD247 expression presented as MFI, gating on CD3 cells. FIG. 7E is a schematic representation showing the percentage of circulating CD11b⁺CD33⁺HLA-DR⁻ cells in CRC-patients before (control) and after 5-fluoruracil (5-FU/FOLFOX treatment in 6 patients. FIG. 7F is a schematic representation showing the expression of CD247 in T-cells in CRC-patients before (control) and after 5FU/FOLFOX treatment in 6 patients. FIG. 7G is a schematic representation showing the percentage of circulating CD11b⁺CD33⁺HLA-DR⁻ cells in CRC-patients before (control) and after FOLFIRI treatment in 4 patients. FIG. 7H is a schematic representation showing the expression of CD247 in T-cells in CRC-patients before (control) and after FOLFIRI treatment in 4 patients. Dashed lines represent the normal mean values of % MDSCs (FIGS. 7E and 7G) and CD247 levels (FIGS. 7F and 7H) that were measured in gated CD3⁺ cells and are presented as the expression in the experimental group relative to the mean of expression in healthy donors (as 100%);**, P<0.01; ***, P<0.001.

FIG. 8A is a schematic representation of the mouse model for CRC. FIG. 8B is a schematic representation of a kinetic study of Gr1⁺CD11b⁺ (mice MDSC) accumulation. Blood samples were collected from mice during CRC development and progression at the indicated time points, and tested for MDSC accumulation by flow cytometry. FIGS. 8C and 8D are schematic representations of MDSC accumulation within the colons as evaluated by flow cytometry analysis. The Graphs represent the absolute number of MDSCs within the lamina propria (FIG. 8C) and the epithelium (FIG. 8D); *, P<0.05; ***, P<0.001; ns=non-significant. FIGS. 8E and 8F are schematic representations of the levels of MDSC isolated from the lamina propria (FIG. 8E) and epithelium (FIG. 8F) of the colons from each experimental group as analyzed by flow cytometry. The graphs (means of triplicates±s.e.m., n=4) are representative of a typical experiment out of three independent performed;**, P<0.01; ns=non-significant. FIGS. 8G and 8H are schematic representations of NO⁻ production levels. The lamina propria (FIG. 8G) and epithelium (FIG. 8H) fractions isolated from the colons of CRC-mice were analyzed for NO⁻ production by flow cytometry analysis gaiting on the MDSC population. Graphs represent production levels, as shown by MFI. All in vivo experiments involved 6 mice per group and were repeated three times yielding similar results. Graphs (means of triplicates±s.e.m., n=6) are representative of a typical experiment out of three performed. *, P<0.05; ***, P<0.001; ns=non-significant.

FIG. 9A is a photograph showing representative spleens of the different experimental groups. FIGS. 9B and 9C are schematic representations of MDSCs accumulation as measured in the spleen by flow cytometry analysis, showing the percentage (FIG. 9B) and absolute numbers (FIG. 9C). FIG. 9D is a schematic representation of the percentage of T regulatory cells in spleens from CRC-mice. Levels of Tregs from each experimental group were evaluated after fixation/permeabilization and double staining for CD4⁺Foxp3⁺. Graph (means of triplicates±s.e.m., n=4) is representative of a typical experiment out of three performed. FIGS. 9E and 9F are schematic representations of NO⁻ (FIG. 9E) and ROS (FIG. 9F) production. Splenocytes isolated from normal, CRC and 5FU-, irinotecan (CPT11)- and 5FU/CPT11-treated CRC-mice were analyzed for NO⁻ and ROS production by flow cytometry analysis, gating on MDSCs. Graphs represent mean fluorescence intensity (MFI). FIGS. 9G-9I are schematic representations showing NO⁻ and ROS production in cultured MDSCs as analyzed by flow cytometry gaiting on CD11b⁺Gr1⁺ MDSCs (9G), or CD11b⁺Ly6C^{high}Ly6G monocytic MDSCs (M-MDSCs) and CD11b⁺Ly6C^{low}Ly6G⁺ granulocytic MDSCs (G-MDSCs) sub-populations (9H and 9I). FIG. 9J is a schematic representation showing the percent of proliferating T cells compared to steady-state levels of non-activated cells in each group. FIG. 9K is a schematic representation showing CD8+ cell proliferation. Splenocytes were labeled with CFSE and activated with anti-CD3 and anti-CD28 antibodies or left non-activated. The proliferative response was assessed by monitoring cell divisions of gated CFSE-labeled CD8⁺ T-cells. The percent of proliferating cells was calculated and compared to steady-state levels of non-activated cells in each group. FIG. 9L is a schematic representation showing the expression level of CD247. Splenocytes from the experimental groups were analyzed for CD247 expression levels indicated by MFI, gating on CD3⁺ cells and. All in vivo experiments involved 6 mice per group and were repeated three times yielding similar results. Graphs (means of triplicates±s.e.m., n=6) are representative of a typical experiment out of three performed. FIG. 9M is a schematic representation showing the expression level of CD247. Splenocytes from the experimental groups were analyzed for CD247 expression levels gating on CD8⁺ cells and indicated by MFI. Graphs (means of triplicates±s.e.m., n=5) are representative of a typical experiment out of three independent performed. 5FU and CPT11 display opposite effects on MDSC accumulation and CD247 expression in the colons of CRC-mice. FIGS. 9N and 9O are schematic representations showing the expression level of CD247. Cells were isolated from the lamina propria (FIG. 9N) and epithelium (FIG. 9O) of the colons from each experimental group and analyzed by flow cytometry for CD247 expression levels (MFI), gating on CD3⁺ cells. Graphs (means of triplicates±s.e.m., n=4) are representative of a typical experiment out of three independent performed. *, P<0.05;**, P<0.01; ns=non-significant.

FIG. 10A is a schematic representation of the CRC mouse model including treatment timelines. FIG. 10B is a schematic representation showing MDSCs accumulation as measured in PBLs by flow cytometry analysis. The graph represents the percent of MDSCs in each experimental group. FIG. 10C is a photograph showing representative colon structures. FIG. 10D is a schematic representation showing Kaplan-Meyer curve (n=20) of CRC, 5FU-treated, CPT11-treated, 5FU/CPT11-treated or MDSC-depleted CPT11-treated CRC-mice. All in vivo experiments involved 6 mice per group and were repeated three times yielding similar results. Graphs (means of triplicates±s.e.m., n=6) are representative of a typical experiment out of three performed; **, P<0.01; ***, P<0.001.

FIGS. 11A-I are schematic representations showing levels of cleaved caspase 3. Splenic MDSCs from each group were analyzed for the expression of cleaved caspase-3 by flow cytometry analysis gating on MDSCs (FIG. 11A). FIG. 11B shows the effect of 5FU and FIG. 11C shows the effect of CPT11 on cleaved caspase-3 expression. FIG. 11D shows the effect of the drugs on primary MDSCs. FIG. 11E shows the effect of the drugs on differentiated CD11c⁺CD11b⁺DCs. FIG. 11F shows the effect of the drugs on F4/80⁺CD11b⁺ macrophages. All experiments involved 6 mice per group and were repeated three times yielding similar results. Graphs (means of triplicates±s.e.m., n=6) are representative of a typical experiment out of three performed. *, P<0.05; ***, P<0.001; ns=non-significant. FIG. 11G shows cleaved caspase-3 expression (MFI) in cultured MDSCs using flow cytometry analysis gaiting on CD11b⁺Ly6C^{high} Ly6G monocytic MDSCs (M-MDSCs) and CD11b⁺Ly6C^{low}Ly6G⁺ granulocytic MDSCs (G-MDSCs) sub-populations. FIGS. 11H and 11I show cleaved caspase-3 expression (MFI) in T (CD3⁺) lymphocytes (FIG. 11H) and in B (B220⁺) lymphocytes (FIG. 11I). All ex vivo experiments involved 5 and in vivo experiments 6 mice per group and were repeated three times yielding similar results. Graphs (means of triplicates±s.e.m., n=6) are representative of a typical experiment out of three performed.

FIG. 12A is a schematic representation showing S100A8/9 mRNA levels and FIG. 12B is a schematic representation showing S100A8/9 protein levels in MDSCs isolated from the spleen of CRC-mice, or CRC-mice treated with 5FU or CPT11. FIG. 12C is a schematic representation showing mRNA levels of S100A8/9 in the colon of CRC-mice treated with 5FU or CPT11 relative to the expression in untreated CRC-mice as evaluated by Real Time PCR analysis. FIGS. 12D-12G are schematic representations of the levels of various cell populations: CD11c⁺CD11b⁺DCs (FIG. 12D), F4/80⁺CD11b⁺ macrophages (FIG. 12E), CD11c+MHCII+CD80+ cells (FIG. 12F) and F4/80⁺MHCII+CD80+ cells (FIG. 12G). FIGS. 12H-12K are schematic representations of MDSCs isolated from spleens of CRC mice that were ex vivo cultured with 10 ng/ml GM-CSF in the absence or presence of scaled-doses (0, 1.25, 2.5, 5 and 10 μmol/L) of CPT11 (FIGS. 12H and 12I) or 5FU (FIGS. 12J and 12K) for 3 days. The phenotype of differentiated DCs (FIGS. 12H and 12J) and macrophages (FIGS. 12I and 12K) was then evaluated. All in vivo experiments involved 6 mice per group and were repeated three times yielding similar results. Graphs (means of triplicates±s.e.m., n=6) are representative of a typical experiment out of three independent performed. Ex vivo experiments involved 4 mice per group and were repeated three times yielding similar results. Graphs (means of triplicates±s.e.m., n=4) are representative of a typical experiment out of three performed. Data shown are the mean±s.e.m. *, P<0.05; **, P<0.01; ***, P<0.001 analyzed using 2-way ANOVA. FIGS. 12L-12O are schematic representations of mRNA levels of various pro-inflammatory molecules generated by MDSCs. MDSCs isolated from spleens of CRC-mice (n=4) were cultured ex vivo in the presence of scaled-doses (0, 1.25, 2.5, 5 and 10 μmol/L) of 5FU (FIGS. 12M and 12O) or CPT11 (FIGS. 12L and 12N) for 3 days. TNFα (FIGS. 12 L and 12M) and S100A9 (FIGS. 12N and 12O) mRNA levels were evaluated by Real Time PCR analysis performed on the primary MDSCs. Graphs (means of triplicates±s.e.m., n=3) are representative of a typical experiment out of three independent performed; *, P<0.05; **, P<0.01.

FIG. 13A is a schematic representation showing a mouse model for chronic inflammation. FIGS. 13B and 13C are schematic representations of the percentage of MDSCs within the PBLs (FIG. 13B) and the spleen (FIG. 13C). PBLs and spleens from normal, inflamed, inflamed 5FU-treated, CPT11-treated or 5FU/CPT11-treated mice were analyzed for MDSC accumulation by flow cytometry analysis. FIG. 13D is a schematic representation of the absolute number of MDSCs within the spleen of normal, inflamed and inflamed mice treated with 5FU, CPT11 or 5FU/CPT11. FIGS. 13E and 13F are schematic representations showing NO⁻ (FIG. 13E) and ROS (FIG. 13F) production in splenocytes by flow cytometry analysis gating on the MDSC population. Graphs represent production levels, as shown by MFI. FIG. 13G is a schematic representation of cleaved caspase-3 levels. The expression of cleaved caspase-3 was analyzed by flow cytometry, gating on MDSC populations. FIG. 13H is a schematic representation of the percentage of Tregs. Tregs derived from the spleens of each experimental group were evaluated by measuring CD4⁺Foxp3⁺ cells. FIG. 13I is a schematic representation of the percent of T cell proliferation. Splenocytes were labeled with CFSE and activated with anti-CD3 and anti-CD28 antibodies or left non-activated. The proliferative response was assessed by monitoring cell divisions of gated CFSE-labeled Thy1.2⁺ (CD90⁺) T-cells. The percent of proliferating cells was calculated and compared to steady-state levels of non-activated cells in each group. FIG. 13J is schematic representation showing percent CD8+ proliferation of splenocytes. FIGS. 13K-13M are schematic representations showing relative CD247 expression. PBLs from each experimental group were double stained for CD247 (FIG. 13K) and CD3s-chain (FIG. 13L). CD247 expression levels were measured in gated CD3⁺ cells and are presented as the expression in the experimental group relative to normal mice (as 100%). Splenocytes from each group were double stained for CD247 and NCR1. CD247 expression levels were measured in NK (NCR1⁺) cells and are presented as the expression in the experimental group relative to normal mice (as 100%) (FIG. 13M). FIG. 13N is a schematic representation of the percent of specific allogeneic cell clearance. NK-cell mediated clearance of CFSE-labeled allogeneic (CFSE^{low}) and syngeneic (CFSE^{high}) splenocytes was evaluated by monitoring the ratio between CFSE^{low}/CFSE^{high} in the spleen (top) and PBLs (bottom) in each experimental group. The graphs of FIGS. 13A-13C, 13E-13G, 13I, and 13N represent means of triplicates±s.e.m., n=5. The graphs of FIGS. 13D, 13H and 13K-13M represent means of triplicates±s.e.m., n=4). All graphs are representative of a typical experiment out of three independent performed; *, P<0.05; **, P<0.01; ***, P<0.001; n.s.=non-significant.

### DETAILED DESCRIPTION OF EMBODIMENTS

The present invention is based on the surprising finding that the levels of myeloid derived suppressor cells (MDSC) having the profile CD11b⁺CD33⁺HLA-DR⁻ and/or CD11b⁺CD33⁺HLA-DR^{low} in a cancer patient's sample, as measured prior to commencing therapy, can serve as a predictor for the patient's response to therapy.

The present invention therefore provides a combinatorial analysis to evaluate the immune status of cancer patients prior to and following a given therapy, measuring MDSC levels or MDSC levels and their suppressive characteristics. Moreover, testing the patient prior to treatment will depict predictive criteria of whether the patient will respond to the therapy. As will be shown in Example 1 below, based on data on melanoma patients subjected to Ipilimumab treatment, it was shown that if high levels of MDSCs (more then 55%) are detected in the blood, this indicates immunosuppression and predicts, with very high significance, non-responsiveness to treatment and very low survival. Testing MDSCs prior to the given therapy also provides a tool to suggest pre or combined treatment with additional drugs that neutralize the inflammatory and immunosuppressive environment prior to or in conjunction with a given chemo or immune based therapy. This analysis is also performed in different intervals during the therapy to monitor changes in the immune status during treatment to evaluate whether the immune system functions properly; if an increase in MDSC levels (or the levels of their suppressive characteristics) is detected during treatment this indicates that the response to treatment may be reduced and hence the type of treatment, or its continuation can be reconsidered.

Chemotherapeutic treatments, and in particular immunotherapeutic treatments are costly and their success rates are currently poor, partly due to the fact that evaluations of the patients' immune status are not performed prior to and during treatment.

As demonstrated in Example 1 below, showing a clinical experiment analyzing 56 patients with melanoma (stage 4) subjected to Ipilimumab treatment, a significant correlation was found between high levels of MDSCs and low responsiveness to Ipilimumab treatment as reflected by poor survival.

The predictive value of MDSC measurements was also demonstrated in colorectal cancer as shown in Example 2 below. Colorectal cancer (CRC) is associated with chronic inflammation and immunosuppression mediated by myeloid-derived suppressor cells (MDSC). Although chemotherapy reduces tumor burden at early stages, it tends to have limited effect on progressive disease, possibly due to adverse effects on the immune system in dictating disease outcome. As shown in Example 2 advanced CRC patients display enhanced MDSC levels and reduced CD247 expression.

Monitoring the immune status of stage IV CRC patients, prior to and following FOLFOX or FOLFIRI treatments revealed that prior to therapy the patients displayed a suppressed immune status as indicated by the elevated MDSC levels and down-regulated CD247, which is a key molecule that “senses” immune functionality and regulates T- and NK-cell immune responses (10). Recent data demonstrated that 5FU treatment leads to a selective MDSC apoptosis and tumor regression in mice (11).

During chemotherapeutic treatments, while FOLFOX reduced accumulation of circulating MDSCs that was accompanied by up-regulated CD247 expression, FOLFIRI displayed opposite effects, enhancing the suppressive environment.

To gain better understanding of 5-fluoruracil (5FU) and Irinotecan (CPT11) adverse effects on host immunity, a mouse CRC model that mimics the human disease was used (1). Similar to the patients, CRC-mice displayed an immunosuppressive status. As shown in Example 2, CPT11 but not 5FU increases immunosuppression by inducing MDSC insensitivity to apoptosis, arresting their differentiation and retaining their suppressive features. Moreover, 5FU/CPT11 combined treatment displays harmful effects, resulting in a dysfunctional immune response associated with cancer progression and short survival, showing that CPT11 antagonizes the anti-cancer activity of 5FU by exerting its detrimental immunoregulatory effects.

These results highlight the importance of developing therapeutic regimens that can target both the immune system and the tumor in developing improved personalized treatments for cancer.

As used in the specification and claims, the singular forms “a”, “an” and “the” include plural references unless the context clearly dictates otherwise.

The invention therefore provides in one of its aspects a method for predicting a cancer patient's response to treatment with a therapeutic agent, said method comprising the steps of:
(a) Measuring the amount of myeloid derived suppressor cells (MDSC) having the profile CD11b⁺CD33⁺HLA-DR⁻ and/or CD11b⁺CD33⁺HLA-DR^{low}, in at least one biological sample obtained from the patient; and
(b) Determining whether the amount of MDSC of said profile is higher than a predetermined standard value or higher than a control sample;
   wherein
(i) said therapeutic agent is a chemotherapeutic agent or an immunotherapeutic agent or a combination thereof, and wherein
(ii) detection of a high amount of MDSC of the above profile in the at least one biological sample as compared with the predetermined standard value or the control sample indicates that the patient will not respond or will poorly respond to treatment with said agent.

In another aspect, the present invention provides a method for monitoring a cancer patient's response to treatment with a therapeutic agent, said method comprising the steps of:
(a) Measuring the amount of myeloid derived suppressor cells (MDSC) having the profile CD11b⁺CD33⁺HLA-DR⁻ and/or CD11b⁺CD33⁺HLA-DR^{low}, in at least one biological sample obtained from the patient; and
(b) Determining whether the amount of MDSC of said profile is higher than a predetermined standard value or higher than a control sample;
   wherein
(i) said therapeutic agent is a chemotherapeutic or immunotherapeutic agent or a combination thereof, and wherein detection of a high amount of MDSC of the above profile in the at least one biological sample as compared with the predetermined standard value or the control sample indicates that the patient is not responding or is poorly responding to treatment.

The methods disclosed herein are applicable to any type of cancer patient. Cancers in accordance with the invention include, but are not limited to adrenal cancer, bladder cancer, brain cancer, breast cancer, cervical cancer, colorectal carcinoma, endometrial cancer, gastro-intestinal cancers, head and neck squamous cell carcinoma, leukemia, malignant lymphoma, including Hodgkin's lymphoma, liver cancer, melanoma, ovarian cancer, pancreatic cancer, prostate cancer, renal cancer, sarcoma, small cell lung cancer, non-small cell lung cancer, and thyroid cancer.

In a specific embodiment the cancer is melanoma.

In a specific embodiment, the present invention provides a method for predicting a melanoma patient's response to treatment with ipilimumab.

In another specific embodiment the cancer is colorectal carcinoma.

As used herein the term “treating” or “treatment” of a disease in a patient refers to administration of a “therapeutic agent” intended for preventing one or more of the disease symptoms, inhibiting disease development, stabilizing its progression, or ameliorating or delaying the appearance of one or more of its symptoms.

The “therapeutic agent” in the context of the present invention is a chemotherapeutic agent or an immunotherapeutic agent. Chemotherapeutic agents and immunotherapeutic agents are compounds that are well known in the art and their selection depends on the cancer being treated. This selection is well within the skill of an attending doctor.

Suitable chemotherapeutic agents include, but are not limited to, antimetabolite drugs, DNA alkylating agents, platinum compounds, enzyme inhibitors (e.g. topoisomerase inhibitors, tyrosine kinase inhibitors), vincalkaloids, taxanes, receptor antagonists, and antibiotics. The invention also pertains to chemotherapeutic combination therapies. Including by not limited to the combinations FOLFOX and FOLFIRI.

FOLFOX is a chemotherapy regimen for treatment of colorectal cancer, made up of the drugs folinic acid (leucovorin), fluorouracil (5-FU) and Oxaliplatin.

FOLFIRI is a chemotherapy regimen for treatment of colorectal cancer, made up of the drugs folinic acid (leucovorin), fluorouracil (5-FU) and irinotecan.

Studies comparing the FOLFOX and the FOLFIRI regimens indicated that in some cases FOLFOX is superior since it leads to higher overall survival rates (12, 13). However, other studies have demonstrated equal efficacy for these treatments (14).

A “DNA alkylating agent” is an agent which attaches an alkyl group to DNA and thereby prevents its replication. Such agents are well known in the art and are used to treat a variety of tumors. Non-limiting examples of DNA alkylating agents are Nitrogen mustards, such as Cyclophosphamide, Mechlorethamine, Uramustine, Melphalan, Chlorambucil, Ifosfamide, Bendamustine; Nitrosoureas, such as Carmustine, Lomustine, Streptozocin; Alkyl sulfonates, such as Busulfan; and ThioTEPA.

“Platinum compounds” are a subclass of the DNA alkylating agents. Non-limiting examples of such compounds include Cisplatin, Carboplatin, Nedaplatin, Oxaliplatin, Satraplatin, and Triplatin tetranitrate.

“Topoisomerase inhibitors” are agents that interfere with the action of topoisomerase enzymes (topoisomerase I and II). Topoisomerases are enzymes that control the changes in DNA structure by catalyzing the breaking and rejoining of the phosphodiester backbone of DNA. Such agents are well known in the art. Non-limiting examples of Topoisomerase I inhibitors include CPT-11/Irinotecan, topotecan, camptothecin, and lamellarin D.

Irinotecan (CPT-11, CPT11) is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

Non-limiting examples of Topoisomerase II inhibitors include, but are not limited to, Etoposide, Teniposide, Anthracyclines (e.g. Doxorubicin, Daunorubicin), Mitoxantrone, amsacrine, aurintricarboxylic acid, ellipticines and HU-331 a quinolone synthesized from cannabidinol.

“Tyrosine kinase inhibitors” (TKIs) are a class of chemotherapy medications that inhibit, or block, the enzyme tyrosine kinase. Non limiting examples of TKI chemotherapeutic agents include: Imatinib (brand name: Gleevac), gefinitib (Iressa) [which have been approved by the Food and Drug Administration for use in humans] Erlotinib (Tarceva), Lapatinib (Tykerb), Sunitinib (Sutent), Sorafenib (Nexavar), Nilotinib (Tasinga), Bosutinib, Neratinib, and Vatalanib.

“Antimetabolite drugs” include pyrimidine antagonists, purine antagonists and folic acid analogues.

Non limiting examples of pyrimidine antagonists include: 5-fluoruracil (5-FU, 5FU), arabinosylcytosine (cytarabine), capecitabine (an oral 5-FU pro-drug), gemcitabine and decitabine.

5-FU is a pyrimidine base containing a fluoride atom at the 5 carbon position on the ring. Uracil is a naturally occurring pyramidine base used in nucleic acid synthesis. It is converted to thymidine by enzyme action. 5-FU is similar in structure to uracil and is converted to two active metabolites (FdUMP and FUTP) that inhibit the activity of the enzyme thymidylate synthetase. The enzyme normally converts uracil to thymidine by adding a methyl group at the fifth carbon of the pyrimidine ring. 5-FU mimics the natural base and functions to inhibit DNA synthesis. The carbon group cannot be added because of the fluoride atom at position 5. Normal DNA synthesis fails. dUTP and FdUTP are incorporated into DNA so that it cannot function normally. In addition, FUTP is incorporated into RNA leading to faulty translation of the RNA. Thus, the synthesis of multiple forms of RNA (messenger, ribosomal, transfer and small nuclear RNAs) is blocked. These combined actions on DNA and RNA are cytotoxic to the rapidly dividing cancer cells.

5-FU is used for the treatment of many malignancies: breast, head and neck, adrenal, pancreatic, gastric, colon, rectal, esophageal, liver and G-U (bladder, penile, vulva, prostate).

Non limiting examples of purine class antimetabolites include: Fludarabine or 2-fluoro-ara-amp, and 6-Mercaptopurine (6-MP).

Folate antagonists generally function by impeding enzyme action. Non limiting examples of the folic acid class antimetabolites include: methotrexate and Pemetrexed.

Leucovorin (Folinic acid) is a reduced folic acid and is used as an adjuvant in cancer therapy, for example in combination with other chemotherapy drugs (e.g. 5-FU) to improve efficacy of the chemotherapeutic agent or as a “chemoprotectant”. This compound has the chemical designation of L-Glutamic acid N[4[[(2-amino-5-formyl1,4,5,6,7,8hexahydro4oxo6-pteridinyl)methyl]amino]b-enzoyl]-, calcium salt (1:1).

Information regarding cancer therapeutic agents and known therapeutic combinations can be found in the US National Cancer Institute's web site http://www.cancer.gov/ or in the US National Comprehensive Cancer Network's web site, http://www.nccn.org/.

Cancer patients are subjected to various types of immune-based therapies since most tumors display immunogenic features. The aim of such therapies is to increase the function of the patients' immune system by blocking inhibitory and/or apoptotic receptors expressed on immune cells such as T cells or by controlling inhibitory signaling pathways and also to boost the immune system by immunization protocols or transfer of ‘educated’ anti-tumor T lymphocytes. Examples for the first strategy are the Ipilimumab treatment (for example in melanoma), which is an anti-CTLA4 antibody that neutralizes the inhibitory stage of the T lymphocytes aiming at increasing the immune system functionality, the lambrolizumab treatment, which is an anti-PD1 antibody and the MPDL3280A treatment which is an anti-PD-L1 (the ligand of PD1) antibody. PD1-PD-L1 interactions also provide negative/apoptotic signals that dampened response of the immune system against the tumor. Blocking the harmful effects of these inhibitory receptors is supposed to induce recovery of the patient's immune function. However, if there are additional key factors that suppress the immune system, which dominate the environment, the suggested treatment will not succeed. Examples for the second strategy include immunization protocols using autologous or heterologous tumor cells with or without dendritic cells, and boosting the patient's immune system by transferring activated ‘educated’ anti-cancer T cells (TILs) in the presence or absence of cytokines and/or reagents used in the first strategy (adoptive T cell transfer). Again, a suppressive environment generated in the patient during tumor development will inhibit efficient responses to such immune boosting therapies.

Therefore, suitable immunotherapeutic agents according to the invention include, but are not limited to immunomodulatory agents including for example, therapeutic antibodies, immune checkpoints inhibitors, cytokines (e.g. IL-2 or interferon α), T cell modulators, tumor infiltrating lymphocytes (TIL), and therapeutic or preventive cancer vaccines.

“Therapeutic antibodies” in the context of the present invention relate to different types of monoclonal antibodies that are used in cancer treatment. Such antibodies are directed against tumor markers or antigens and exert their activity either by inducing antibody-dependent cell cytotoxicity (ADCC), or by association with a toxic agent. Non limiting example of therapeutic antibodies include: Anti-CD52 (Alemtuzumab, Campath®), Anti-HER2/neu (erbB2) receptor (Trastuzumab, Herceptin®), anti-HER1/EGFR (Cetuximab, Panitumumab); Anti-VEGF-A (Bevacizumab); Anti-CD20 (Rituximab, Tositumomab, Ibritumomab); and Anti-CD33 (Gemtuzumab).

The therapeutic antibodies also include immune checkpoint blocking antibodies such as anti-CTLA4 (e.g. Ipilimumab), anti-PD-1 (e.g. lambrolizumab) and anti-PD-L1 (the ligand of PD1) (e.g. MPDL3280A).

“Immune checkpoints inhibitors” are compounds that interfere in pathways that regulate the immune status of a patient. The immune system depends on several checkpoints to avoid activity of the immune system on healthy cells. Tumor cells often take advantage of these checkpoints to escape detection by the immune system. Examples of immune checkpoints are CTLA-4 and PD-1.

In specific embodiments said immunotherapeutic agent is Ipilimumab, lambrolizumab or their combination.

“Ipilimumab” (also known as MDX-010, MDX-101 or Yervoy) relates to a humanized anti-CTLA4 monoclonal antibody. By targeting CTLA4, Ipilimumab neutralizes the inhibitory stage of cytotoxic T lymphocytes and thereby activates these cells of the immune system and allows them to successfully target and destroy cancer cells. Ipilimumab is presently used in the treatment of melanoma patients.

“lambrolizumab” relates to a humanized anti-PD1 monoclonal antibody.

“Tumor infiltrating lymphocytes” (TILs) are a type of white blood cells found in tumors. TILs are implicated in killing tumor cells, and the presence of lymphocytes in tumors is often associated with better clinical outcomes. TILs may be used as an adoptive cell transfer therapy to treat cancer. For example, autologous lymphocytes are isolated from patients' tumors and grown to very large numbers of cells in vitro. Prior to TIL treatment, patients are given nonmyeloablative chemotherapy to deplete native lymphocytes that can suppress tumor killing. Once lymphodepletion is completed, patients are then infused with TILs in combination with interleukin 2 (IL-2).

“Cancer vaccines” stimulate the immune system's ability to fight cancer. Preventive (or prophylactic) vaccines are intended to prevent cancer from developing in healthy subjects, while treatment (or therapeutic) vaccines are intended to treat an existing cancer. A non-limiting example of a cancer vaccine is the antigen presenting cells (APC)-based sipuleucel-T (Provenge®) for treating metastatic prostate cancer.

The invention also provides methods for treating a cancer patient comprising as an initial step determining whether the cancer patient would be responsive to treatment with the therapeutic agent as described above. If the patient is found to be responsive, the therapeutic agent is administered.

Therefore, the present invention provides a method of treating a cancer patient with a therapeutic agent, said method comprising, or alternatively, said method consisting of, the steps of:
(a) Determining whether said patient is responsive to the therapeutic agent as described above; wherein said therapeutic agent is a chemotherapeutic agent or an immunotherapeutic agent or a combination thereof; and
(b) wherein the patient was determined to be responsive, administering to the patient an effective amount of said chemotherapeutic agent or said immunotherapeutic agent or a combination thereof.

An “effective amount” of a chemotherapeutic agent or an immunotherapeutic agent or a combination thereof is an amount sufficient to effect beneficial or desired results, i.e. an amount sufficient to treat, ameliorate, alleviate, inhibit disease development, stabilize disease, prevent or reduce symptoms of cancer in a cancer patient. An effective amount can be administered in one or more administrations, applications or dosages. Such delivery is dependent on a number of variables including the time period for which the individual dosage unit is to be used, the bioavailability of the chemotherapeutic or immunotherapeutic agent, the route of administration, etc. It is understood however that specific dose levels of the chemotherapeutic or immunotherapeutic agents in accordance with this aspect of the invention for any particular patient depends upon a variety of factors including the activity of the specific compound employed, the route of administration, the age of the patient, its gender, body weight, general health, additional drugs that the patients receives etc. Determining the precise dosages is well within the physician's skill.

The chemotherapeutic or immunotherapeutic agent can be administered by any route as known in the art, for example by oral administration or by parenteral administration including intramuscular, intravenous or subcutaneous administration. In some embodiments, when oral administration is employed the agent is administered using a convenient daily dosage regimen, and may be in the form of tablets, pills, capsules, semisolids, powders, sustained release formulations, solutions, suspensions, aerosols or any other appropriate composition.

The chemotherapeutic or immunotherapeutic agent is preferably administered in a pharmaceutical composition that may further comprise suitable pharmaceutical grade excipients and carriers. Such excipients and carriers include, but are not limited to, starch, cellulose, talc, glucose, lactose, sucrose, gelatin, malt, glycerol, propylene glycol, water, ethanol, oils of various origins, sodium chloride, saline, aqueous dextrose, and the like.

In one embodiment the method of the invention is used for determining the patient's therapeutic regime by at least one of the following:
(a) Refraining from treatment with said chemotherapeutic agent, said immunotherapeutic agent or the combination thereof;
(b) Discontinuing treatment with said chemotherapeutic agent, said immunotherapeutic agent or the combination thereof; or
(c) Combining the treatment with said chemotherapeutic agent, said immunotherapeutic agent or the combination thereof with at least one additional compound being an anti-inflammatory and/or an anti-MDSC therapeutic agent.

As used herein an “anti-inflammatory agent” relates to a compound that reduces inflammation. Anti inflammatory agents include, but are not limited to, NSAIDs (non-steroidal anti-inflammatory drugs) including broad-spectrum NSAIDs (which non-specifically inhibit both COX-1 and COX-2) e.g. aspirin, sulindac, ibuprofen, piroxicam, and COX-2 specific agents, such as celecoxib, corticosteroids (e.g. the glucocorticoids dexamethasone, hydrocortisone and prednisone), and antibodies directed against cytokines associated with inflammation, e.g. TNFα.

As used herein an “anti-MDSC therapeutic agent” relates to a compound that inhibits MDSC. Such a compound may act by deactivation of MDSC, differentiation of MDSC into mature cells, blocking development of MDSC, or depletion of MDSC. Deactivation of MDSC may be achieved by using nitric oxide (NO) inhibitors, phosphodiesterase-5 (PDE-5) inhibitors, Nitro-aspirins, L-NAME (N (G)-Nitro-L-Arginine Methyl Ester), Arginase inhibitors, COX2 inhbitors, NOHA, ROS inhibitors (e.g. synthetic triterpenoids), MDSC migration inhibitors (e.g. anti glycan antibodies and CSF-1R inhibitors), histamine inhibitors or anti IL-17 antibodies. Differentiation of MDSC into mature cells can be achieved by using vitamins (e.g. ATRA (all trans retinoic acid), Vitamin A, vitaminD3, IL-12 or CpG. Blocking development of MDSC can be achieved by using bisphosphonates (e.g. zoledronic acid), or by modulating cell signaling using JAK2/STAT3 inhibitors, multi-kinase inhibitors or VEGF inhibitors. Depletion of MDSC can be achieved for example by using cytotoxic agents (e.g. gemcitabine, cisplatin, paclitaxel or 5-FU), HSP 90 inhibitors (e.g. 17-DMAG), IL-6R blockers or antibody drug conjugates (Wesolowsli et al., J. for Immuno Therapy of Cancer 2013 1:10). In addition, MDSC depletion can be achieved using antibodies directed against MDSC cell markers, for example anti CD33 antibodies. For research purposes MDSC depletion in mouse models can be achieved for example by using anti Gr1 antibodies.

As used herein the term “therapeutic regimen” or “therapeutic regime” relates to a regulated scheme of treatment. This scheme of treatment comprises for example administering a therapeutic agent to a patient at specific intervals, either alone or in combination with additional therapeutic agents or treatment modalities (e.g. irradiation). In the context of the present invention, an “altered” therapeutic regimen or “altering” the therapeutic regimen relates herein to a change in the schedule of treatment which is brought about by the acquired information on the immune status of the patient, as exemplified in the levels of MDSC in a patient's biological sample. The change in schedule of treatment may be a decision to refrain from treating the patient with a chemotherapeutic or immunotherapeutic agent (e.g. Ipilimumab), or, in case that the patient is already being treated with a chemotherapeutic or immunotherapeutic agent, the change may be a decision to cease treatment. Alternatively, an altered therapeutic regimen may relate to a decision to include at least one additional therapeutic agent in the therapeutic regimen, a therapeutic agent which is directed to enhancing the patient's immune system and may be an anti inflammatory drug and/or a drug directed at the elimination of MDSC.

As used herein the terms “response”, “responsiveness”, “responsive” or “responder” to treatment with a chemotherapeutic or immunotherapeutic agent refers to an improvement in at least one relevant clinical parameter as compared to an untreated subject diagnosed with cancer, or as compared to the clinical parameters of the same subject prior to said treatment.

The term “therapeutic failure”, “non responder”, “non-responsive” or “not respond” to treatment with a chemotherapeutic or immunotherapeutic agent, refers to a treated cancer patient not experiencing an improvement in at least one of the clinical parameters. This term also encompasses a poor response to therapy which indicates a very low level of response which is not clinically significant or sufficient. For example, low responsiveness to a chemotherapeutic or immunotherapeutic agent treatment may be reflected by poor survival.

The methods of the invention include the step of measuring the amount of myeloid-derived suppressor cells (MDSC) having the profile HLA DR⁻ CD33⁺CD11b⁺ or HLA DR^{low} CD33⁺CD11b⁺ in a biological sample obtained from the patient.

“MDSC” are a heterogeneous population of early myeloid progenitors, immature granulocytes, macrophages, and dendritic cells at different stages of differentiation. These cells have the capacity to suppress both the cytotoxic activities of natural killer (NK) and NKT cells, and the adaptive immune response mediated by CD4⁺ and CD8⁺ T cells. Human MDSCs commonly express Siglec-3/CD33 and lack lineage markers and HLA-DR, but heterogeneous expression of other cellular markers indicate that multiple subsets exist. Multiple positive markers used to identify MDSC are known in the art. These include, as non limiting examples, expression of CD33, CD14, CD15, CD66b, or CD11b.

The MDSC of the present invention are characterized as having the phenotype HLA DR⁻ CD33⁺CD11b⁺ or HLA DR^{low} CD33⁺CD11b⁺.

“HLA-DR” is a MHC class II cell surface receptor encoded by the human leukocyte antigen complex.

“CD11b” is expressed on the surface of many leukocytes including monocytes, granulocytes, macrophages, and natural killer cells.

“CD33” or Siglec-3 is a trans-membrane receptor expressed on cells of myeloid lineage. CD33 is usually considered myeloid-specific, but it can also be found on some lymphoid cells.

The presence of HLA DR CD33⁺CD11b MDSC in the biological sample is determined using detecting agents which can be antibodies, specifically, anti HLA-DR antibodies, anti CD11b antibodies and anti CD33 antibodies. Wherein the presence or absence of each of the cell markers is denoted by a + or a − sign, respectively. Thereby HLA DR CD33⁺CD11b MDSC are MDSC which lack HLA-DR, and express CD33 and CD11b. The designation HLA DR^{low} relates to MDSC which show a relatively low expression level of HLA-DR. When staining a cell population that comprises MDSC with anti-HLA DR antibodies, three cell populations can be identified according to the intensity of staining—HLA DR^{high}, HLA DR^{low} and HLA DR^{negative}. The present invention relates to the cell populations that show negative and/or low staining with anti HLA-DR antibodies. The definitions of “high”, “low” and “negative” staining levels are relative in each tested sample and are well within the knowledge of the skilled person in the art of the invention.

As indicated above, in certain embodiments the detecting agents can be antibodies. Antibodies may be prepared using methods well known in the art (see for example Harlow and Lane, Antibodies: A Laboratory Manual, Cold Spring Harbor Laboratory, New York (1988)).

The term “Antibody” as used herein refers to IgG, IgM, IgD, and IgA antibodies. This term refers to whole antibodies or fragments of the antibodies comprising the antigen-binding domain, e.g. scFv, Fab, F (ab′) 2, bi-specific antibodies, diabodies, and other fragments capable of binding to the target molecule. The definition includes polyclonal antibodies and monoclonal antibodies. The monoclonal antibodies can be derived from various species such as murine, rabbit, goat or rat. Non limiting examples of commercial antibodies that can be used to identify the HLA DR⁻ CD33⁺CD11b MDSC of the invention are provided in the Examples section below.

As used herein a “high level” or “higher amount” of MDSC detected in a patient's sample relates to a percent of cells that is significantly (e.g. as determined by statistical determination) higher than a predetermined standard value or significantly higher than a control sample. A “control sample” relates to a sample obtained from a healthy subject, i.e. a subject that does not suffer from a disease associated with chronic inflammation and is known to have a functional immune system. “Standard” or a “predetermined standard” as used herein, denotes either a single standard value or a plurality of standards with which the level (percent of) MDSC in the tested sample is compared. The standards may be prepared by determining the level of MDSC present in a sample obtained from a plurality of healthy subjects. After such standards are prepared, it is possible to compare the level of MDSC obtained from a specific tested subject to the corresponding value of the standards, and thus obtain an assaying tool.

More specifically, in certain embodiments, wherein “higher” or “high” levels of MDSC are indicated, it is meant that MDSC are present in between about 5% to 100%, more specifically about 10%, or about 20%, or about 30%, or about 40%, or about 50%, or about 60%, or about 70%, or about 80%, or about 90%, higher amounts than in the control sample or the predetermined standard.

Non limiting examples of higher levels of MDSC in patients' samples are demonstrated in the Examples below. In a specific embodiment a high level of MDSC in a sample relates to a percent value of above 50% MDSC per total cells in the sample, preferably above 55% of the cells in the sample.

As used herein the percent of MDSC relates to the percent of MDSC having the profile CD33⁺CD11b⁺ within the HLA DR population of cells in the tested biological sample, or the percent of MDSC having the profile CD33⁺CD11b⁺ within the HLA DR⁻ and the HLA DR^{low} population of cells in the tested biological sample

The presence of a high level of MDSC in the cancer patient's sample indicates that the patient is immune suppressed and therefore predicts a poor response to treatment with the therapeutic agent, at least as a single agent. However, since the patient is identified as being immune suppressed the therapeutic regimen offered to the patient may be altered by including therein in addition to the cancer therapeutic agent, additional therapeutic agents capable of counteracting the detrimental effects of MDSC.

Additional parameters may be measured and combined with the level of MDSC in order to determine the patient's response to therapy.

The additional parameters include measuring the level of LDH (lactate dehydrogenase) prior to or during therapy, measuring the level of various suppressive characteristics of the patient and assessing the metastatic status of the patient.

“LDH” (lactate dehydrogenase) is an enzyme found in nearly all living cells. LDH catalyzes the conversion of pyruvate to lactate and back, as it converts NADH to NAD⁺ and back. Detection techniques for LDH are well known in the art, using for example enzymatic reactions performed on patients' serum samples or assays that are commercially available.

Relatively high levels of LDH (e.g. levels higher than 450 U/I, 480 U/I, or 500 U/I, preferably higher than 480 U/I) are predictors of a shorter survival time of the cancer patient.

Similarly, a more advanced metastatic state of the patient is a predictor of a shorter survival time of the cancer patient.

As used herein “suppressive characteristics” of MDSC denote various measurable features or molecules that can be used as surrogates to determine the suppressive state of the MDSC. Such characteristics include, but are not limited to the expression levels of LDH, S100A8 and/or S100A9 proteins, the levels of cleaved caspase 3, intracellular nitric oxide (NO), reactive oxygen species (ROS), iNOS, and arginase 1, levels and activity in said biological sample; and suppressive activity of the MDSC on T cells. In certain embodiments, MDSC suppressive activity on T cells is measured by assessing down regulation of CD247 and/or SNX9 expression, and/or impaired T cell proliferation.

“S100A8” is a calcium- and zinc-binding protein which plays a prominent role in the regulation of inflammatory processes and immune response. It can induce neutrophil chemotaxis and adhesion. S100A8 functions involve proinfammatory, antimicrobial, oxidant-scavenging and apoptosis-inducing activities.

“S100A9” is a calcium-binding protein also known as migration inhibitory factor-related protein 14 (MRP14) or calgranulin B. It is encoded by the S100A9gene.

These proteins are predominantly found as calprotectin (a complex of S100A8/A9) which has a wide plethora of intra- and extracellular functions. Detection of S100A8 and/or S100A9 can be done using antibodies which are specific for these proteins. Such antibodies are commercially available for example from R&D Systems. mRNA encoding these proteins can be detected using appropriate probes, e.g. as shown in the Examples.

“Caspase-3” (CASP3) is a caspase protein that interacts with caspase-8 and caspase-9. It is encoded by the CASP3 gene. The CASP3 protein is a member of the cysteine-aspartic acid protease (caspase) family. Sequential activation of caspases plays a central role in the execution-phase of cell apoptosis. Caspases exist as inactive proenzymes that undergo proteolytic processing at conserved aspartic residues to produce two subunits, large and small, that dimerize to form the active enzyme. This protein cleaves and activates caspases 6 and 7; and the protein itself is processed and activated by caspases 8, 9, and 10. Detection of the cleaved form of caspase 3 hence indicates that the cell is undergoing apoptosis. Detection of cleaved caspase 3 can be done using antibodies which are specific for the cleaved form. Such antibodies are commercially available for example from R&D Systems, sigmaAldrich etc.

“Intracellular Nitric oxide” (NO) is an important cellular signaling molecule involved in many physiological and pathological processes. “Reactive oxygen species” (ROS) are chemically reactive molecules containing oxygen. Examples include oxygen ions and peroxides. ROS are formed as a natural byproduct of the normal metabolism of oxygen and have important roles in cell signaling and homeostasis. An increase in ROS levels may result in significant damage to cell structures.

Detection techniques for NO or ROS are well known in the art, using for example assays that are commercially available. For example, ROS may be detected using ROS Brite™ APF which is a fluorogenic probe to measure hydroxyly radical in cells using conventional fluorescence microscopy, high-content imaging, microplate fluorometry, or flow cytometry. The cell-permeant ROS Brite™ APF reagent is nonfluorescent and produces bright green fluorescence upon reaction with hydroxyl radical. The resulting fluorescence can be measured using fluorescence imaging, high-content imaging, microplate fluorometry, or flow cytometry. NO may be detected using a cell permeable analog of DAF-2 that is hydrolyzed to DAF-2 by intracellular esterases. This agent can be used in fluorescence microscopy to measure real-time changes in nitric oxide (NO) levels. Exemplary protocols for measuring NO and ROS are provided in the Examples.

“iNOS” (inducible NO Synthase) is an enzyme catalyzing the production of NO from L-arginine and is a member of the Nitric oxide synthases family. It is an inducible NO synthase and is involved in immune response, binds calmodulin at physiologically relevant concentrations, and produces NO as an immune defense mechanism, as NO is a free radical with an unpaired electron. Detection of iNOS can be done for example using antibodies which are specific for this enzyme. Such antibodies are commercially available (e.g. by R&D Systems).

“Arginase” (also termed arginine amidinase, canavanase, L-arginase, arginine transamidinase) is a manganese-containing enzyme. It is the final enzyme of the urea cycle. Detection of Arginase 1 can be done for example using antibodies which are specific for this enzyme. Such antibodies are commercially available (e.g. by SigmaAldrich).

“CD247” (Cluster of Differentiation 247) refers to T-cell surface glycoprotein CD3 zeta chain also known as T-cell receptor T3 zeta chain. Detection of CD247 on the surface of T cells can be done using antibodies which are specific for this marker. Such antibodies are commercially available. An example for CD247 detection technique is provided in the Examples.

The “SNX9” (Sorting nexin-9) protein is a member of the sorting nexin family. Members of this family contain a phox (PX) domain, which is a phosphoinositide binding domain, and are involved in intracellular trafficking. Detection of SNX9 in T cells can be done using antibodies which are specific for this marker. Such antibodies are commercially available. An example for SNX9 detection technique is provided in the Examples.

Elevated levels of NO, elevated levels of ROS, elevated levels of S100A8 and/or S100A9 proteins, low levels of cleaved caspase 3, and MDSC suppressive activity on T cells indicate that the patient suffers from an immune compromised state which affects or will affect the patient's responsiveness to therapy.

As used herein a “biological sample” refers to any cell containing sample obtained from the patient. The sample can be a peripheral blood sample including whole blood or fractionated blood, a spleen biopsy, cells obtained from lymph nodes, tissue biopsy, tissue sections (e.g. a colon tissue section) or a tumor sample. The sample may be fresh, previously frozen, preserved or cryopreserved.

Preferably, fresh whole blood samples are analyzed upon lysing the erythrocytes. Methods for cryopreservation of cells and tissues are well known in the art, e.g. by mixing the cells with dimethyl sulfoxide (DMSO). One example of a cryopreservation protocol is provided in the Examples below.

The HLA DR⁻ CD33⁺CD11b MDSC in the biological sample can be detected using fluorescence activated cell sorter (FACS), immunohistology (immunohistochemistry of tissue sections), as well as ELISA, RIA or Western blotting of the suitable MDSC cell markers i.e. CD33, CD11b. In addition, the MDSC in the biological sample can be detected using nucleic acid-based detection assays including in situ hybridization, RT-PCR (real-time polymerase chain reaction) and Northern blotting with probes specific for the MDSC markers, i.e. CD33, CD11b.

A first step in some of the detection methods requires staining of the cells with the relevant antibodies. Methods for staining cells with antibodies are well known in the art and usually the appropriate protocols are included in the instructions that accompany any commercially available antibody. An example of a staining procedure is provided in the Examples below. Briefly, the cells (e.g. in the whole blood sample) are placed in suitable plates, washed with staining buffer, incubated with the antibodies (preferably labeled antibodies), followed by additional washing. For intracellular markers, the cells are fixed prior to staining (e.g. with paraformaldehyde) and permeabilized (with a permeabilization buffer, comprising e.g. PB-0.1% saponin, and 1% human serum.

The detection of the HLA DR⁻ CD33⁺CD11b MDSC in accordance with the invention may be performed by flow cytometry in a fluorescence activated cell sorter (FACS). Protocols for carrying out FACS analysis are well known in the art. FACS provides a method for analysis and sorting a heterogeneous mixture of cells in a biological sample, one cell at a time, based upon the specific light scattering and fluorescent characteristics of each cell. In general, a first step in flow cytometry is obtaining cells in suspension, labeling the cells with fluorescently labeled agents (preferably antibodies) which bind specifically the desired markers. The cell may be labeled as a whole living cell, or permeabilized prior to labeling (using a fixative) in order to allow labeling of intracellular molecules. The labeled cells are than subjected to flow analysis in the FACS. A non-limiting example of a protocol for carrying out a FACS analysis is provided in the Examples below.

The detection of the HLA DR⁻ CD33⁺CD11b MDSC in accordance with the invention may be also performed by immunohistochemistry of tissue sections. Protocols for carrying out immunohistochemistry are well known in the art. In general, a first step in immunohistochemistry is the preparation of tissue sections. For example, a tissue biopsy potentially comprising the cells is fixed (e.g. using formalin or paraformaldehyde), the fixed biopsy is embedded in a paraffin block and thin tissue sections or slices are prepared (e.g. in a microtome). Alternatively, the tissue biopsy is frozen in liquid nitrogen and thin tissue sections or slices are prepared (e.g. using a cryostat). The tissue sections are than stained with labeled detecting agents (e.g. antibodies). The label in such cases may be enzymatic. A non-limiting example of a protocol for carrying out immunohistochemistry is provided in the Examples below.

Dot blot, slot blot, Western blot and ELISA analyses are commonly used assays for detecting the presence and amount of target molecules (usually proteins) in a sample. In a dot blot assay the whole sample which putatively includes the target molecule is placed as such onto a porous substance (e.g. a nitrocellulose membrane) and the proteins within the sample are immobilized in the membrane in the form of a dot.

In a Western blot assay, the sample which putatively includes the target molecule is first run on a separating gel thereby the proteins are separated one from the other and form a gradient according to their size. Next, the separated proteins are blotted onto a nitrocellulose membrane and immobilized onto the membrane according to their respective position in the gel.

In an ELISA (Enzyme-linked immunosorbent assay) the whole sample which putatively includes the target molecule is placed as such onto a non-porous substance (e.g. a well of a standard 96 well plate) and the proteins within the sample coat the bottom of the well. Alternatively, sandwich ELISA assay may be performed. In such case, the bottom of the well is pre-coated with specific primary antibodies directed against a target molecule. Hence the target molecule is immobilized onto the surface via specific binding to these antibodies. Next, the presence and amount of the target molecule is assessed by an immunoassay conventionally employing a first and a second antibody, whereby the second antibody is detectably labeled so as to detect the presence of the target molecule and its amount.

The detection in accordance with the invention is performed using detecting molecules. The term “detecting molecules” as used herein refers to any molecule that can specifically recognize the MDSC markers of the invention or any one of the suppressive molecules. The detecting molecules may be amino acid molecules or nucleic acid molecules. Non limiting examples of detecting molecules include antibodies, receptors, ligands, substrates or nucleic acid probes. The detecting molecules may be detectably labeled.

The term “detectably labeled” as used herein refers to a detecting molecule which is associated with a compound that may be detected by an appropriate reaction (enzymatic or color reaction) or by fluorescent excitation and assists in visualizing, quantifying or detecting the target molecule (the labeling agent).

The labeling agent may be a fluorescent compound, e.g. fluorescein (or a fluorescein derivative such as FITC) or phycoerythrin (PE), a fluorescent particle such as quantum dot, an enzyme such as horseradish peroxidase (HRP) or alkaline phosphatase (AP), a chromophore, or an electrochemically active or a radioactive molecule.

It should be emphasized that the detectably labeled detecting molecules are non-naturally occurring molecules.

The term “Association” or “associated” as used herein refers to any physical or chemical forces such as Van-der-Walls, coordinative, covalent, ionic, electrostatic, dipole-dipole, or hydrogen association (bond or interaction). The association may occur directly or indirectly (i.e. comprising one or more intermediate agents). In some embodiments the intermediate agent is another protein, ligand or spacer. For example, the intermediate agent may be streptavidin, biotin, immunoglobulin binding protein (e.g. protein A, protein G, Protein A/G, protein L, etc.), DNA or RNA molecule, peptide tag or its chelating complex (e.g. polyhistidine tag or metal complex of nitrilotriacetic acid such as Ni-NTA).

The detecting molecules in accordance with the present invention may also be immobilized on a substrate, e.g. a membrane, a filter, beads.

The substrate may be a porous substrate. As used herein, the term “porous substrate” refers to a substrate having a plurality of pores (depressions). These pores have inner voids of the same or varying volume and shape, defined by inner surface. In certain embodiments the substrate pores are nanometric in size, namely having a mean size smaller than 1,000 nm. In some embodiments, the mean pore size is below 500 nm. In other embodiments, the mean pore size is below 300 nm. In further embodiments, the mean pore size is below 200 nm. In other embodiments, the mean pore size is below 100 nm. In other embodiments, the mean pore size is below 50 nm. In further embodiments, the mean pore size is between 300 nm and 50 nm. In specific embodiments the porous substrate is a membrane having 200 nm or 450 nm pores.

The substrate may also be a non-porous substrate.

The substrate may be a flexible or a rigid or a soft substrate, and may be composed of any material. In some embodiments the substrate is a layered substrate, e.g., a porous layer on a non-porous layer or soft layer (such as gel or tissue) on metal layer (holder). The substrate (or one of its layers) may be composed of insulating, conducting or semiconducting material. In some embodiments the substrate may be composed of glassy, polymeric, ceramic, fibrous material, or any combination thereof. In some embodiments the substrate's material may be composed of glass, paper, wool, fleece, gel, cellulose, or any combination thereof. In some embodiments the substrate is composed of a nitrocellulose or PVDF membrane. The nitrocellulose or PVDF membrane may have varying pore sizes, e.g. 0.2 m (200 nm) or 0.45 m (450 nm).

As indicated above, in certain embodiments the method further comprises measuring the expression level of certain suppressive features or markers. These include, but are not limited to LDH, S100A8 and/or S100A9 proteins, cleaved caspase 3, intracellular nitric oxide (NO), reactive oxygen species (ROS), iNOS, and arginase 1, CD247 and/or SNX9. The expression levels of the above noted suppressive markers in the biological sample can be detected using fluorescence activated cell sorter (FACS), immunohistology (immunohistochemistry of tissue sections), as well as substrate based detection assays such as ELISA, RIA or Western blotting or by using nucleic acid-based detection assays including in situ hybridization, RT-PCR and Northern blotting with probes specific for these markers.

The terms “level of expression” or “expression level” are used interchangeably and generally refer to the amount of a polynucleotide or a protein in a biological sample. According to the invention “expression” of a polypeptide, for example S100A8 and/or S100A9 proteins, may refer to transcription into a polynucleotide, translation into a protein, or even posttranslational modification of the protein.

It should be noted that the expression level is reflected by measurement and determination of an expression value. As used herein, the term “expression value”, “level of expression” or “expression level” refers to numerical representation of a quantity of a gene product, which herein is a protein, but may also be an mRNA.

As used herein the term “comparing” denotes any examination of the amount or percent of MDSC or the expression level of any one of the suppressive characteristics of MDSC obtained in the samples of the invention as detailed throughout in order to discover similarities or differences between the measured values and a predetermined standard value or a value obtained in a control sample. It should be noted that comparing according to the present invention encompasses the possibility to use a computer based approach.

In one embodiment the present invention provides a method for predicting a cancer patient's response to treatment with a therapeutic agent, said method consisting of the steps of:
(a) Measuring the amount of myeloid derived suppressor cells (MDSC) having the profile CD11b⁺CD33⁺HLA-DR⁻ and/or CD11b⁺CD33⁺HLA-DR^{low}, in at least one biological sample obtained from the patient; and
(b) Determining whether the amount of MDSC of said profile is higher than a predetermined standard value or higher than a control sample;
wherein
   (i) said therapeutic agent is a chemotherapeutic agent or an immunotherapeutic agent or a combination thereof, and wherein
   (ii) detection of a high amount of MDSC of the above profile in the at least one biological sample as compared with the predetermined standard value or the control sample indicates that the patient will not respond or will poorly respond to treatment with said agent.

In another embodiment, the present invention provides a method for monitoring a cancer patient's response to treatment with a therapeutic agent, said method consisting of the steps of:
(a) Measuring the amount of myeloid derived suppressor cells (MDSC) having the profile CD11b⁺CD33⁺HLA-DR⁻ and/or CD11b⁺CD33⁺HLA-DR^{low}, in at least one biological sample obtained from the patient; and
(b) Determining whether the amount of MDSC of said profile is higher than a predetermined standard value or higher than a control sample;
wherein
   (i) said therapeutic agent is a chemotherapeutic or immunotherapeutic agent or a combination thereof, and wherein
   (ii) detection of a high amount of MDSC of the above profile in the at least one biological sample as compared with the predetermined standard value or the control sample indicates that the patient is not responding or is poorly responding to treatment.

The invention further encompasses kits, e.g. pre-packages diagnostic kits, such as those described below comprising ampoules or vessels containing:
(a) Detecting molecules specific for MDSC having the profile CD11b⁺CD33⁺HLA-DR⁻ and/or CD11b⁺CD33⁺HLA-DR^{low}; and optionally further comprising at lease one of the following:
   i) detecting molecules for determining LDH levels;
   ii) detecting molecules specific for at least one of S100A8, S100A9, cleaved caspase 3, iNOS, arginase 1, NO, ROS, CD247 or SNX9;
   iii) secondary agents and/or buffers for performing detection of MDSC, LDH and at least one of S100A8, S100A9, cleaved caspase 3, iNOS, arginase 1, NO or ROS; and
(b) instructions for use.
   wherein said kit is for use in a method for predicting a cancer patient's response to treatment with a chemotherapeutic agent or an immunotherapeutic agent or a combination thereof, or for use in a method for determining whether the therapeutic regimen of a cancer patient should be altered.

In a specific embodiment, the detecting molecules specific for MDSC having the profile CD11b⁺CD33⁺HLA-DR⁻ and/or CD11b⁺CD33⁺HLA-DR^{low} comprise at least one antibody directed against each one of CD11b⁺, CD33⁺ and HLA-DR. The antibody may be but is not limited to a mouse antibody, a rabbit antibody, a goat antibody etc.

In a specific embodiment, the detecting molecules specific for MDSC having the profile CD11b⁺CD33⁺HLA-DR⁻ and/or CD11b⁺CD33⁺HLA-DR^{low} comprise at least one probe or primer nucleic acid capable of recognizing each one of the nucleic acids encoding CD11b⁺, CD33⁺ and HLA-DR.

Optionally, the kit further comprises detecting molecules specific for at least one of LDH, S100A8, S100A9, cleaved caspase 3, iNOS, arginase 1, NO or ROS.

In a specific embodiment, the detecting molecules specific for at least one of LDH, S100A8, S100A9, cleaved caspase 3, iNOS, or arginase 1 comprise at least one antibody directed against each one of these molecules.

In a specific embodiment, the detecting molecules specific for at least one of LDH, S100A8, S100A9, cleaved caspase 3, iNOS, or arginase 1, comprise at least one probe or primer nucleic acid capable of recognizing each one of the nucleic acids encoding each one of these molecules.

In a specific embodiment, the detecting molecules specific for at least one of LDH, NO or ROS comprise suitable reagents for functional detection of each one of these molecules.

The term “secondary agent” as used herein refers to an agent that binds to a detecting molecule and as such it binds indirectly to the target molecule via the detecting molecule, or to an agent that is necessary for performing the detection assay e.g. an enzymatic detection assay.

Non-limiting examples of secondary agents include antibodies, immunoglobulin binding proteins (e.g. protein A, protein G, Protein A/G, protein L), streptavidin (that binds to biotin and biotin labeled compounds), DNA or RNA strands that bind to complementary DNA or RNA strands, and chelating complexes (such as Ni-NTA) that bind to specific peptide tags (e.g. polyhistidine tag).

In the case of detecting molecules which are antibodies (primary antibodies), the selection of the type of secondary agent (e.g. a secondary antibody) is dependent on the class of the primary antibody (e.g. IgG or IgD), and on the source of the primary antibody, e.g. if the detecting antibody is a mouse antibody, the secondary antibody would be an anti-mouse antibody. The secondary antibody may be detectably labeled. Non-limiting examples of labels include an enzyme (e.g. HRP or AP), a fluorescent compounds (e.g. fluorescein or phycoerythrin), a chromophore, or an electrochemically active or a radioactive molecule.

In a specific embodiment, the invention provides a kit comprising:
an ampoule containing anti CD11b antibodies, and
an ampoule containing anti CD33 antibodies, or an ampoule containing a mixture of anti CD11b antibodies and anti CD33 antibodies; and
at least one additional ampoule containing a detecting agent specific for at least one of LDH, S100A8, S100A9, cleaved caspase 3, iNOS, arginase 1, NO, ROS, CD247 or SNX9; and
instructions for use.

In a further embodiment the anti CD11b antibodies, the anti CD33 antibodies and the at least one additional detecting agent specific for at least one of LDH, S100A8, S100A9, cleaved caspase 3, iNOS, arginase 1, NO, ROS, CD247 or SNX9 are detectably labeled.

In a further embodiment the anti CD11b antibodies, and/or the anti CD33 antibodies and/or the at least one additional detecting agent specific for at least one of LDH, S100A8, S100A9, cleaved caspase 3, iNOS, arginase 1, NO, ROS, CD247 or SNX9 are mobilized onto a substrate, e.g. a membrane, a filter or a bead.

In another embodiment the invention provides a kit consisting of:
(a) Detecting molecules specific for MDSC having the profile CD11b⁺CD33⁺HLA-DR⁻ and/or CD11b⁺CD33⁺HLA-DR^{low}; and optionally further comprising at lease one of the following:
   i) detecting molecules for determining LDH levels;
   ii) detecting molecules specific for at least one of S100A8, S100A9, cleaved caspase 3, iNOS, arginase 1, NO, ROS, CD247 or SNX9;
   iii) secondary agents and/or buffers for performing detection of MDSC, LDH and at least one of iNOS, arginase 1, NO or ROS; and
(b) instructions for use.
   wherein said kit is for use in a method for predicting a cancer patient's response to treatment with a chemotherapeutic agent or an immunotherapeutic agent or a combination thereof, or for use in a method for determining whether the therapeutic regimen of a cancer patient should be altered.

As used herein, the term “comprising” is intended to mean that the methods and kits include the recited elements, but not excluding others.

The term “consisting of” when used to define methods or kits, shall mean excluding other elements of any essential significance to the method or kit.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3rd ed. Cold Spring Harbor Laboratory Press: Cold Spring Harbor, N.Y.; Sambrook et al., (1989) Molecular Cloning: A Laboratory Manual. 2nd ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4th ed. Wiley-Liss; among others.

### EXAMPLES

The present invention is described further below in working examples which are intended to further describe the invention without limiting the scope therein.

### Example 1: Analysis of Melanoma Patients

Material and Methods

MDSC Analyses:

The human MDCS phenotype as described herein, HLADR-CD33⁺CD11b⁺, was determined using flow cytometry of blood samples, immunohistology of tumor sections, and ELISA. Suppressive features of MDSC were analyzed by measuring NO, ROS, the S100A8/9 pro-inflammatory proteins, cleaved caspase 3 and MDSC suppressive activity on T cells (down regulating CD247 and SNX9 expression and impairing T cell proliferation). Suppressive features are characterized by elevated levels of NO and ROS, increased levels of the S100A8/9 pro-inflammatory proteins, low levels of cleaved caspase 3 and MDSC suppressive activity on T cells (down regulating CD247 and SNX9 expression and impairing T cell proliferation).

Cryopreservation and Thawing Procedure:

Peripheral blood (whole blood) samples were tested as fresh upon lysing the erythrocytes, or were cryopreserved by mixing the sample with DMSO/FCS (freezing medium) and were transferred into cryovial tubes at different volumes (100, 200, 300 and 500 μl/vial). The cryovial tubes were first stored in liquid N₂ containers. For analysis, whole blood samples were then thawed into 15 ml tubes containing preheated medium (RPMI-1640). After one wash whole blood samples were resuspended in PBSX1 (50-100 μl whole blood in 200 μl PBSX1) and stored in 4° C. refrigerators for maximum 1 h prior to staining and flow-cytometry analysis or functional assays.

Antibodies for Surface and Intracellular Markers:

the following labeled anti-human monoclonal antibodies (mAbs) were used for staining: anti-HLA-DR-FITC, anti-CD27-FITC, anti-CD33-PE, anti-CD56-PE, anti-CD11b-APC, anti-CD3ε-APC, anti-HLA-DR-Pacific-blue (Biolegend). Rabbit polyclonal anti-human Cleaved caspase-3-Alexa Fluor 488 was purchased from Cell Signaling Technology. Prior to staining, each antibody was tittered to determine its optimal dilution using cryopreserved whole blood samples obtained from healthy donors.

Staining Whole Blood Samples:

for human myeloid derived suppressor cells (MDSCs) staining, whole blood samples were loaded on a 96U shape plates and washed twice with a flow stain buffer (PBSX1, 1% human serum). After adding anti-CD11b, CD33 and HLA-DR mAb (50 μl/well in flow stain buffer), whole blood samples were incubated for 30 min at room temperature (RT) in the dark. After incubation with mAb 200 μl of 1X eBioscience-Step Fix/Lyse solution was added, mixed thoroughly and incubated for another 30 min at RT in the dark, washed twice and resuspended in 200 μl of flow stain buffer. For CD247 or cleaved caspase-3 intracellular detection, whole blood samples were loaded on 96U shape plates and washed twice with PBSX1. Samples were fixed with 2% paraformaldehyde in PBS for 20 min at 4° C. in the dark, washed twice with PBSX1 and permeabilized (with permeabilization buffer, PB-0.1% saponin, and 1% human serum) for 10 min at RT in the dark. After washing the samples twice anti-CD56, CD3ε, CD247 or anti-CD33, CD11b, HLA-DR and Cleaved caspase-3 antibodies were added (50 μl/well in PB) for 30 min at 4° C. in the dark. Samples were then washed twice with PB and were resuspended with 200 μl flow stain buffer.

Flow Cytometry:

Samples were analyzed by FACSCalibur using Cell Quest software (BD). For MDSCs detection, after the initial FSC/SSC discrimination, the gate was set on HLA-DR⁻ cells and then on the CD11b⁺CD33⁺ population. To evaluate cleaved caspase-3 expression in MDSCs, the gate was first set on HLA-DR⁻ cells and then the percent and mean fluorescent intensity (MFI) of positive cells in the CD11b⁺CD33⁺ population was determined. For CD247 expression in T cells, the gate was first set on CD3e+CD56⁻ cells and then the MFI of CD247 in this population was determined. Cleaved caspase-3 staining was performed using primary Rabbit anti-cleaved caspase-3 (Cell Signaling Technology) and secondary FITC-horse anti-Rabbit antibody (Thermo Scientific).

ROS Detection:

APF (Cell Technology) was used to measure ROS production by MDSC. Whole blood samples were incubated at 37° C. in HBSS in the presence of APF and 5 μg/well LPS for 30 min. Un-stimulated and stimulated samples were then stained with labeled anti-CD33, CD11b and HLA-DR mAb in 50 μl HBSS. After 30 min incubation, 200 μl of 1X eBioscience-Step Fix/Lyse solution was added, mixed thoroughly and incubated for another 30 min at RT. Samples were then washed twice with HBSS and analyzed by four color flow cytometry. Gating was the same as used for Cleaved caspase-3. Aliquots of Antibody-stained samples which were not incubated with APF served as controls.

NO Detection:

DAF-2DA (cell technology) was used to measure NO production by MDSC. Whole blood samples were incubated at 4° C. in PBSX1 in the presence of DAF-2DA for 30 min. Samples were then stained with labeled anti-CD33, CD11b and HLA-DR mAb in 50 μl PBSX1. After 30 min incubation, 200 μl of 1X eBioscience-Step Fix/Lyse solution was added, mixed thoroughly and incubated for another 30 min at RT. Samples were then washed twice with PBSX1 and analyzed by four color flow cytometry. Gating was the same as used for Cleaved caspase-3. Aliquots of Antibody-stained samples, which were not incubated with DAF-2DA served as controls.

Isolation of MDSCs:

Whole blood samples were treated with erythrocyte lysis buffer (ELB-eBioscience) to remove red blood prior to the isolation procedure. White blood cells were first incubated with anti-HLA-DR Ab-coated magnetic beads (Miltenyi) for 30 min and separated using the MACS columns (Miltenyi) according to the manufacturer orders. Negatively selected HLA-DR⁻ cells were then incubated with anti-CD33 coated magnetic beads for 30 min to positively identify CD33⁺HLA-DR⁻ and was followed with another incubation for 30 min with anti-CD11b coated magnetic beads to positively identify CD33 CD11b⁺HLA-DR⁻ MDSCs.

Statistical Analysis:

Statistical analyses were performed using GraphPad Prism 5.04. Averaged values are presented as the mean±s.e.m. When comparing two groups, statistical significance was determined using two-tailed Student's t test. When more than two groups were investigated, we performed an analysis of variance (ANOVA). Survival analyses were assessed using Geham-Breslow-Wilcoxon test. P values of less than <0.05 were considered statistically significant.

Results:

HLA-DR-CD33⁺CD11b⁺ MDSC Levels are Increased in Melanoma Patients.

The percentage of MDSC with the profile CD11b⁺CD33⁺HLA-DR⁻ was measured in peripheral blood samples (3-5 ml) of healthy donors (n=40) and stage-4 melanoma patients (n=56) prior to ipilimumab (anti-CTLA4) treatment. All the melanoma patients were diagnosed with metastatic melanoma. The samples were taken according to the Helsinki approval. Clinical parameters were acquired from the medical records of patients.

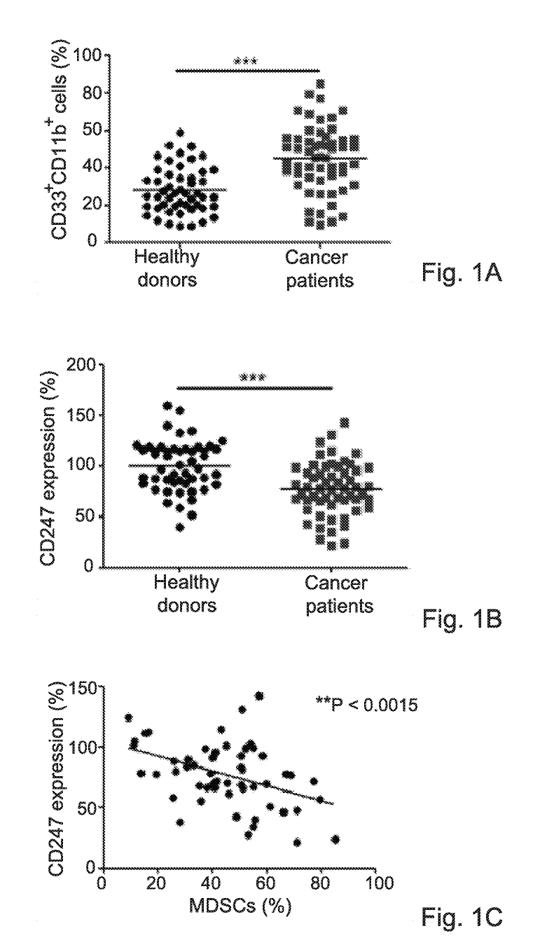

The MDSCs were isolated and analyzed by FACS as described in materials and methods above. As can be seen in FIG. 1A the percentage of MDSC in melanoma patients (the cancer patients) is significantly higher then that observed in healthy donors. Peripheral blood from the healthy donors and the melanoma-patients were also analyzed by flow cytometry for CD247 expression presented as mean fluorescence intensity (MFI), gating on CD3⁺ cells (FIG. 1B). As seen in FIG. 1C there is an inverse correlation between the percentage of MDSCs and CD247 expression presented as % MFI in the melanoma patients. A higher percentage of MDSC correlated with a lower expression of CD247. Down regulation of CD247 is an indication of suppressive activity on T cells.

MDSCs Levels are not Affected by Age, Tumor Origin or Chemotherapeutic Treatments Prior to Ipilimumab Treatments.

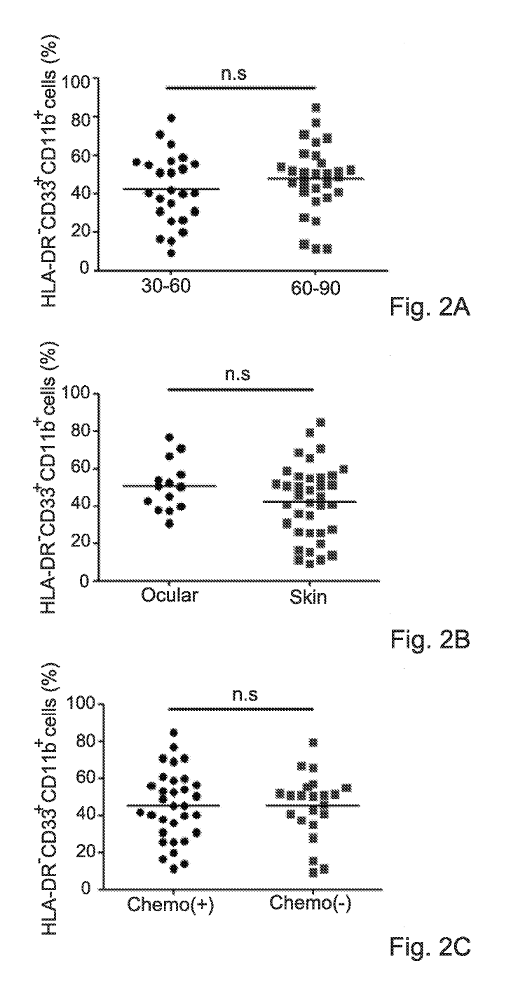

In order to evaluate the effects of additional parameters on the level of MDSC in the blood of melanoma patients, various patient groups were analyzed. First, melanoma patients were divided into two age groups, 30-60 years old and 60-90 years old. Peripheral blood was obtained from the patients and the percentage of MDSCs in the blood was evaluated in these two age groups by using flow cytometry analysis as described in Materials and Methods above. As can be seen in FIG. 1A the age group of the melanoma patient does not affect the level of MDSC in the peripheral blood. Similarly, as shown in FIG. 2B the tumor origin (e.g. skin or ocular) also does not affect MDSC level. Interestingly, also the exposure to chemotherapeutic treatments (prior to ipilimumab treatments) did not seem to affect MDSC levels in these patients.

MDSCs Levels can Distinguish Between Responders and Non-Responders to Ipilimumab Treatment.

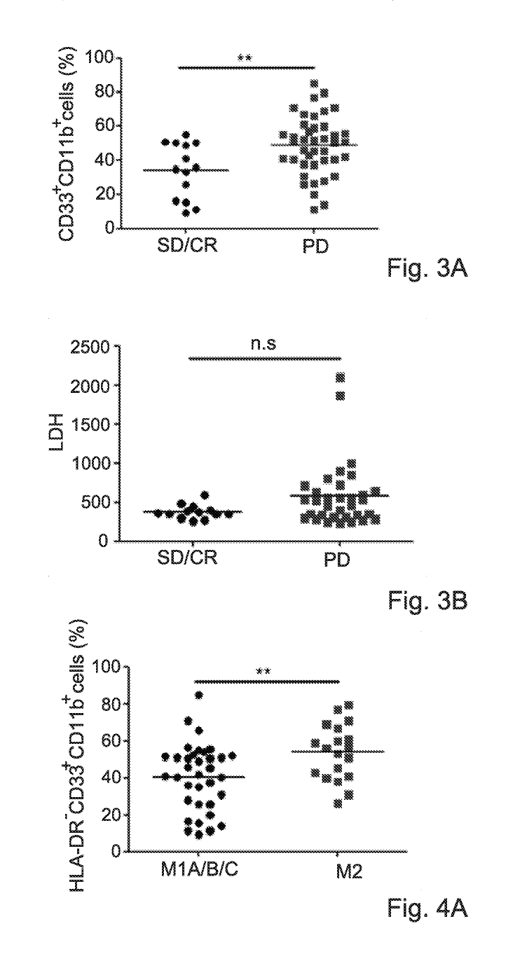

In order to evaluate whether MDSC levels in the blood can predict responsiveness to treatment with the anti CTLA4 antibody ipilimumab, the levels of MDSC in the peripheral blood of melanoma patients were measured, as described above, prior to treatment with ipilimumab. Following treatment with ipilimumab, the melanoma patients were divided into two groups: a group of responders which included patients that manifested stable disease and patients that showed a complete response, and a group of non-responders which included patients with progressive disease. As shown in FIG. 3A there was a strike correlation between high levels of MDSC in the peripheral blood of patients and subsequent failure to respond to treatment with ipilimumab. Clearly, this parameter could distinguish between the two groups of melanoma patients. In addition, the level of lactate dehydrogenase (LDH) was evaluated before the treatment with ipilimumab began. There was no significant difference between the groups of patients with respect to this parameter (FIG. 3B).

High MDSCs Frequencies Correlate with Poor Survival.

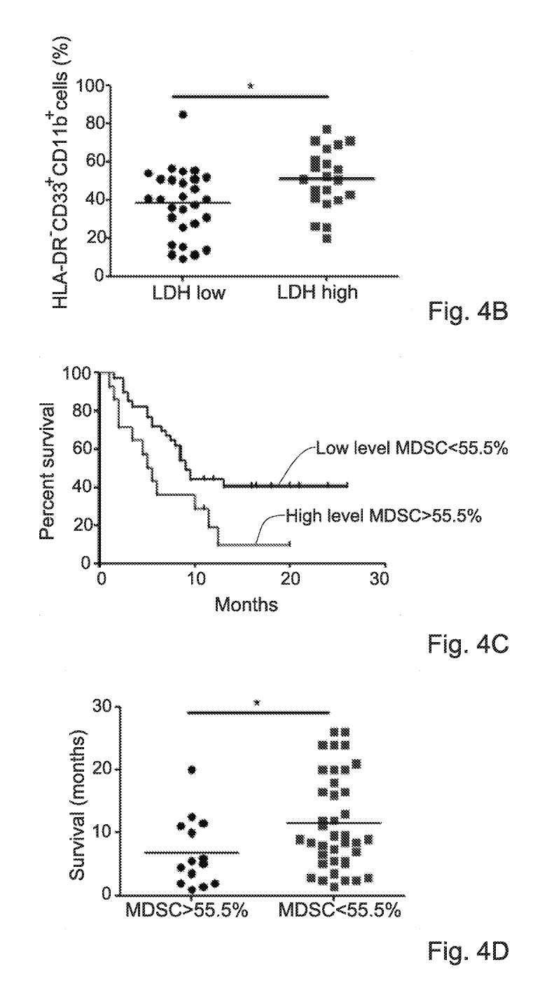

The following experiment was performed in order to evaluate whether MDSC levels in the blood can predict metastatic severity and patient survival. The levels of MDSC, as well as the levels of LDH, were measured in the peripheral blood of melanoma patients, as described above, prior to treatment with ipilimumab. As shown in FIG. 4A, MDSCs levels are higher in patients with higher metastatic severity (M2), as compared with the group with lower metastatic severity (M1A/M1B/M1C). In addition, as shown in FIG. 4B higher MDSCs levels are found in the group of patients with high LDH levels (LDH low<480 U/I; LDH high>480 U/I). In addition, the melanoma patients were divided into two groups according to their MDSCs levels measured prior to the ipilimumab treatments; one group was termed Low MDSCs (<55.5%, n=39) and the other group was termed High MDSCs (>55.5%, n=14). The percent survival and their survival in months were evaluated. As shown in FIG. 4C patients with low MDSCs had a better survival curve than patients with high MDSCs. The survival in months of the Low MDSCs group being 11.56±1.2 (n=39) and of the High MDSCs group being 6.857±1.4 (n=14). Apparently, high MDSC levels are predictors of a more severe metastatic profile and of a shorter survival time of the cancer patient.

LDH Levels and Metastatic Severity Correlates with Patient's Survival.

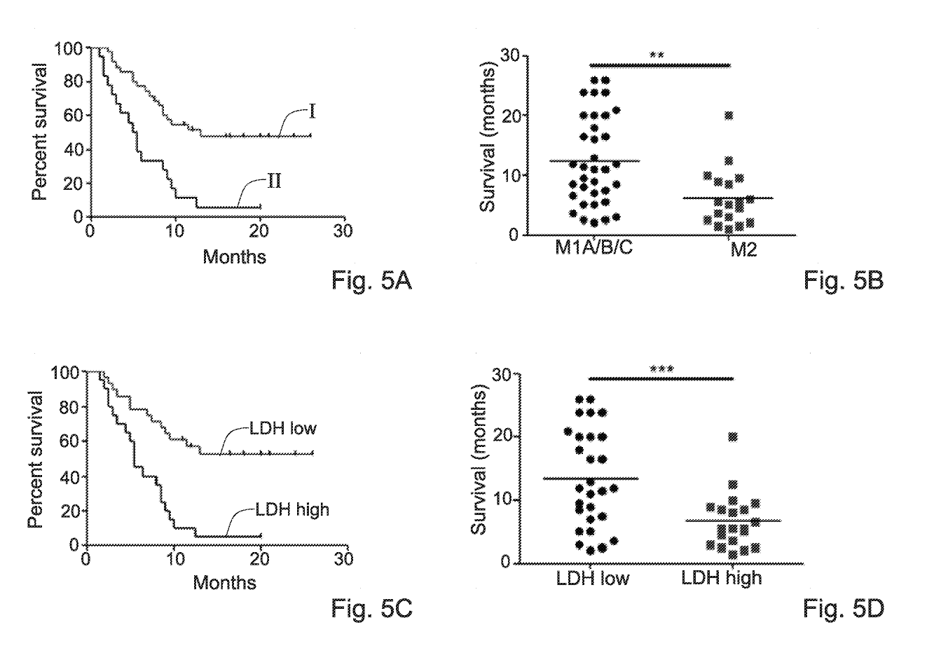

In this experiment the survival of melanoma patients was correlated with the severity of their metastatic stage and with their LDH levels. The patients were divided into two groups according to their metastatic level prior to ipilimumab treatments; the first group included patients with the relatively lower metastatic severity, namely M1A/B/C, and the second group included patients with the higher, M2, metastatic severity. As shown in FIGS. 5A and 5B the survival of patients in the M1A/B/C group was higher than the survival of the patients in the M2 group, being 12.4±1.2 months (n=35) in the M1A/B/C group and 6.1±1.1 months (n=18) in the M2 group. The patients were divided into two groups according their LDH levels prior to ipilimumab treatments; the first group included patients with the relatively lower LDH level, namely LDH<480 U/I; and the second group included patients with the higher LDH level, namely LDH>480 U/I. As shown in FIGS. 5C and 5D the survival of patients in the LDH low group was higher than the survival of the patients in the LDH high group, being 13.5±1.4 months (n=28) in the LDH low group and 6.6±0.9 months (n=20) in the LDH high group. Apparently, high LDH levels and a severe metastatic stage are predictors of a shorter survival time of the cancer patient.

A Combination of MDSCs, LDH and Metastatic Levels Before Ipilimumab Treatments Distinguishes Between High and Low Survival Rates of Patients.

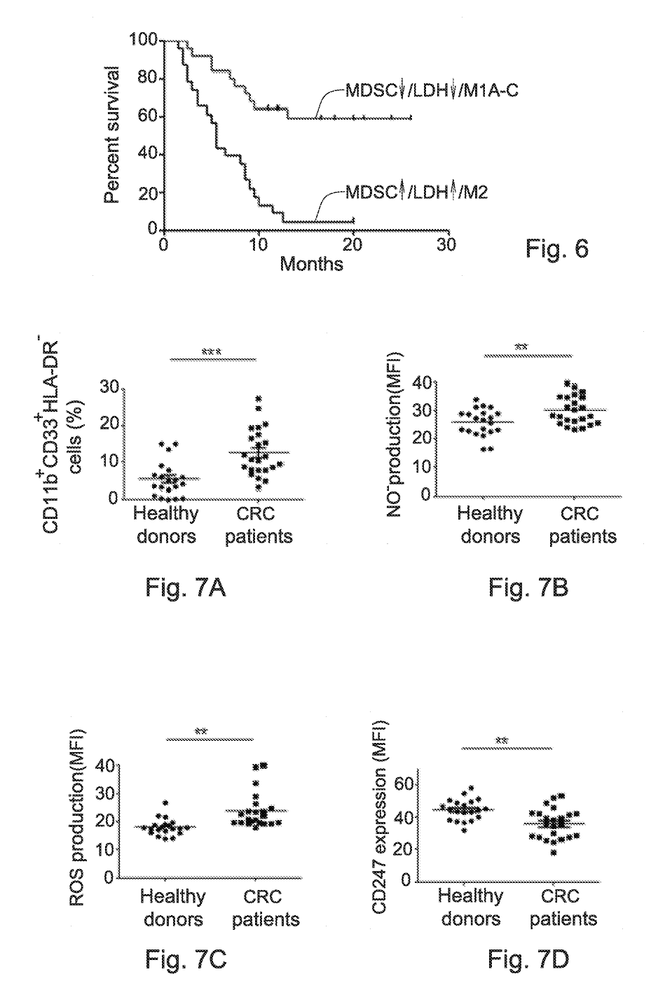

In view of the results shown in the two previous sections, in the following experiment the combined predictive value of measuring MDSC levels, LDH levels and metastatic severity on patients' survival was analyzed. Melanoma patients were divided into two groups according to their initial parameters as measured before the first ipilimumab treatment. Group I included patients with low levels of MDSCs, low levels of LDH and had metastatic staging of M1A-C (MDSC↓LDH↓M1A-C); group II included patients with high MDSCs levels, high LDH levels and a metastatic staging of M2 (MDSC↑LDH↑M2). The low and high levels were defined as shown in the above examples. As clearly shown in FIG. 6 patients of Group I had a significantly higher survival rate as compared with the patients of Group II. Apparently, high MDSCs levels, high LDH levels and a severe metastatic stage are reliable predictors of a shorter survival time of the cancer patient.

Analysis of Ipilimumab Responders

Table 1 shows parameters of 14 patients that responded to the ipilimumab treatment out of the 56 patients involved in this study. The Table shows the type of response (SD—stable disease; CR—complete response; PR—partial response; PD—progressive disease). Patient's number 12 and 14 had a very short period of responsiveness in comparison to the other responders, as indicated by their low survival rates (in months). These patients had high LDH levels on the day the first ipilimumab treatment was given and their staging level was M1C.

### Example 2: Analysis of Metastatic Colorectal Cancer (CRC) Patients and CRC Mouse Models

Material and Methods

Patients:

Peripheral blood samples were collected from 23 stage IV metastatic CRC-patients prior to and every 2 months in the course of chemotherapy treatments. All patients that were diagnosed with metastatic CRC underwent surgery and were not previously treated with chemotherapy. 20 healthy donors were used as controls. The samples were taken according to the Helsinki approval and analyzed for the indicated immune biomarkers in a “blinded test”, not knowing the therapy specification.

TABLE 1

| | | MDSC | | Survival | | |
|---|---|---|---|---|---|---|
| | Respond | (%) | LDH | (months) | Stage | Status |
| 1 | SD | 55.3 | 259 | 12 | M1A | AWD |
| 2 | CR | 35.8 | 275 | 12 | M1B | AWD |
| 3 | CR | 16.5 | 300 | 24 | M1B | AWD |
| 4 | CR | 48.9 | 340 | 20 | M1B | AWD |
| 5 | CR | 35 | 350 | 16.5 | M1B | NED |
| 6 | SD | 9.2 | 351 | 24 | M1B | AWD |
| 7 | SD | 50.4 | 353 | 20 | M1B | AWD |
| 8 | SD | 41 | 371 | 16.5 | M1B | AWD |
| 9 | SD | 11.5 | 379 | 24 | M1A | AWD |
| 10 | SD | 15.5 | 390 | 26 | M1A | AWD |
| 11 | SD | 50.8 | 442 | 20 | M1B | AWD |
| 12 | SD | 25.8 | 487 | 5.5 | M1C | DOD |
| 13 | PR | 13.8 | Normal | 26 | M1B | AWD |
| 14 | SD-PD | 50.5 | 530 | 8.5 | M1C | DOD |

analyses completion, the specific treatment regiments and clinical parameters were acquired from medical records of the patients and correlation tests were performed.

Mice:

Female C57BL/6 and BALB/c mice (aged 6-8 weeks) were purchased from Harlan (Jerusalem, Israel) and were grown at the Hebrew University specific-pathogen-free facility. All experiments were done in accordance with pre-approved institutional protocols.

In Vivo Mouse Models: A Model for Colorectal Cancer (CRC) and a Model for Tumor-Free Chronic Inflammation:

Mice were injected intraperitoneally with 10 mg/kg body weight of Azoxymethan (AOM, purchased from Sigma-Aldrich (St. Louis, Mo., USA)) dissolved in physiological saline twice in two weeks intervals. Two weeks later, 2% dextran sulfate sodium (DSS, purchased from MP biochemicals Inc. (Santa Ana, Calif., USA)) was given in the drinking water over 7 days, followed by 14 days of regular water (15). This cycle was repeated twice. Animals were sacrificed and analyzed three weeks after the last treatment. To induce a pathology-free chronic inflammation, a previously described protocol subjecting mice to heat-killed Mycobacterium tuberculosis (BCG) treatment (16) was used.

CFSE Staining and Ex-Vivo T-Cell Proliferation Assay:

Splenocytes or purified T-cells isolated by a magnetic column separation system (Miltenyi Biotec) were labeled with 5 M CFSE (Invitrogen) and subjected to TCR-mediated activation as previously described (16).

Ex Vivo Myeloid-Cell Differentiation:

MDSCs were isolated from CRC and control (normal) mice and cultured in the presence or absence of 10 ng/ml GM-CSF (PeproTech) for 3 days. In some samples, 5-fluoruracil (5FU) and Irinotecan (CPT11) were added to the cells, with or without GM-CSF, followed by phenotyping using flow cytometry.

Flow Cytometry Analysis:

Isolated mouse splenocytes and peripheral blood lymphocytes (PBLs) were subjected to cell surface staining as previously described (16), using the following antibodies (Biolegend): FITC-labeled anti-Gr1, and anti-CD11c; PE-labeled anti-F4/80, anti-CD3s, and anti-mNKp46; and biotinylated anti-CD11b detected with streptavidin-Cy5. Intracellular staining for CD247 was performed as previously described (16) by using FITC-labeled anti-CD247 or biotinylated anti-CD247 (lone H146), the latter detected with streptavidin-Cy5. Foxp3 staining was performed according to the manufacturer's instructions (Miltenyi Biotec). Cleaved caspase-3 staining was performed using primary Rabbit anti-cleaved caspase-3 (Cell Signaling Technology, Asp175) and secondary FITC-horse anti-Rabbit antibody (Thermo Scientific). For intracellular NO⁻ and ROS detection, diaminofluoresciein-2 diacetate (DAF-2DA) reagent (NOS 200-1; Cell Technology) and aminophenyl fluorescein (APF) (4011; Cell Technology) were used respectively and determined by flow cytometry analysis.

For human whole blood cell phenotyping, intracellular staining of CD247 cells was performed by first fixing the cells with paraformaldehyde 1% followed by washes and permeabilized with 0.1% saponin. APC-labeled anti-CD11b and anti-CD3, PE-labeled anti-CD33, FITC-labeled anti-HLA-DR and anti-CD247 were used, all purchased from BD Pharmingen and used according to the manufacturer's protocol. After surface staining, cells were treated eBioscience-Step Fix/Lyse solution according to the manufacturer's instructions. All samples were analyzed using FACS Calibur with Cell Quest software (BD).

Cell Isolation from the Colon:

The preparation of single cell suspensions from colons was performed using a modified version of a previously described protocol (17). Briefly, isolated colons were washed with HBSS 5% FBS (Invitrogen), digested, minced, incubated for 15 min at 37° C. and epithelial cell suspension was washed with RPMI. For lamina propria cells, the retained tissue was transferred to collagenase/DNAse (Roche Diagnostic Corporation) solution, incubated for 1 h at 37° C., filtrated and washed with RPMI.

Quantitative PCR Analysis:

Total RNA was recovered from colon cells, splenocytes or isolated MDSCs and subjected to real-time PCR analysis as previously described (16). The sequences of the oligonucleotides used are listed in Table 2.

TABLE 2

| Gene | Forward primer | Reverse primer |
|---|---|---|
| S100a8 | GGAAATCACCATGCCCTCTACAA | ATGCCACACCCACTTTTATCACC |
| | SEQ ID NO: 1 | SEQ ID NO: 2 |
| S100a9 | GGAGCGCAGCATAACCACCATC | GCCATCAGCATCATACACTCCTCA |
| | SEQ ID NO: 3 | SEQ ID NO: 4 |
| Hprt | GTTAAGCAGTACAGCCCCAAA | AGGGCATATCCAACAACAAACTT |
| Hypoxanthine | SEQ ID NO: 5 | SEQ ID NO: 6 |
| Phosphoribosyl- | | |
| transferase | | |
| TNF-α | GCCACCACGCTCTTCTGTCTAC | GGGTCTGGGCCATAGAACTGAT |
| | SEQ ID NO: 7 | SEQ ID NO: 8 |

Western Blot Analysis:

Cells isolated from the spleen or colon were analyzed by Western blotting for the expression of various proteins as previously described (16). The antibodies used for immunoblotting were: anti-S100A9, anti-S100A8, and anti-αTubulin. Specific antibodies were detected by anti-rabbit or anti-goat antibodies conjugated to horseradish peroxidase (Jackson Immunoresearch), followed by enhanced chemiluminescence and exposure at blotting reader (Bio-Rad software).

Histopathology and Immunohistochemistry:

Paraffin-embedded colon tissue sections were prepared from CRC, CRC-5FU or CRC-CPT11 treated and control-untreated mice and stained with hemotoxylin and eosin solution. For immunohistochemistry, after antigen retrieval, sections were incubated at 4° C. with primary antibodies: anti-β-catenin (BD) and anti-Gr-1 (Biolegend). For immunohistochemical staining, universal immuno-peroxidase polymer for mouse tissues (414311F; Histofine) was used, based on a horseradish peroxidase (HRP)-labeled polymer conjugated to anti-Rat. After incubation for 30 min, slide staining was completed by 3-5 min incubation with DAB+Chromogen (Lab Vision), followed by counterstaining with hematoxylin. As a control, samples were stained with each antibody and reagent individually.

Statistical Analysis:

Statistical analyses were performed using GraphPad Prism 5.04. Averaged values are presented as the mean±s.e.m. When comparing two groups, statistical significance was determined using two-tailed Student's t-test. When more than two groups were investigated, an analysis of variance (ANOVA) was performed. Survival analyses were assessed using Fisher's exact test.

For the human experiments, paired t-test was used to compare samples from the same patients before and after FOLFOX or FOLFIRI treatment. Control and CRC groups were investigated by ANOVA.

Results:

FOLFOX and FOLFIRI Therapies of CRC-Patients Display Opposite Effects on CD11b⁺CD33⁺HLA-DR⁻/CD11b⁺CD33⁺HLA-DR^{Low} Myeloid Cells and Immune Status

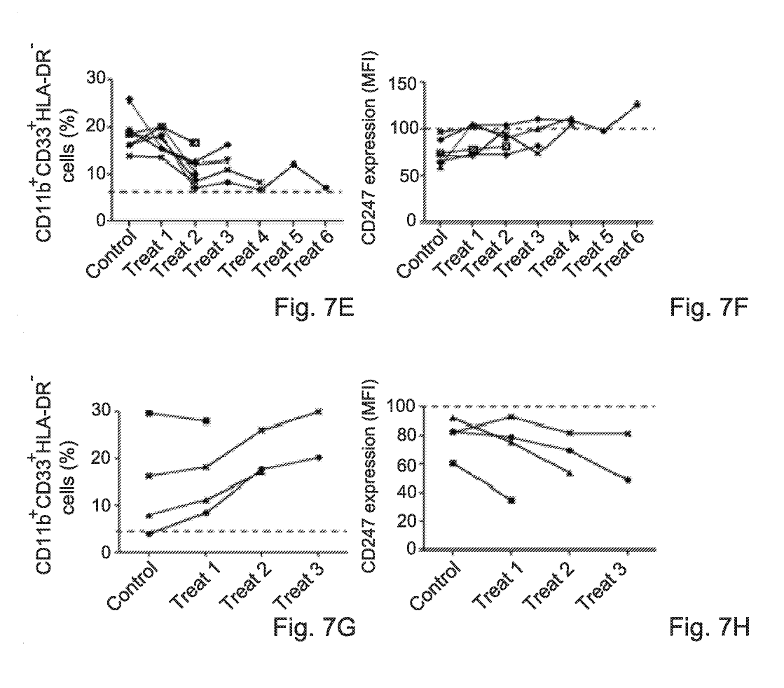

The immune status of 23 stage IV metastatic CRC patients was assessed prior to receiving chemotherapy and compared with the immune status of 20 healthy donors. Peripheral blood samples were obtained from the healthy donors and the CRC patients and the levels of MDSC were measured using flow cytometry analysis of the cells. The percentage of CD11b⁺CD33⁺HLA-DR⁻ MDSCs in the patients' peripheral blood was significantly higher (12.65%+1.35%, p<0.01) as compared to healthy donors (5.35%+1.05%) (FIG. 7A). Moreover a significant increased production of both NO⁻ (FIG. 7B) and ROS (FIG. 7C) by MDSCs was found in CRC patients as compared to healthy donors, showing their immunosuppressive features in the blood of CRC patients. In addition, an inverse correlation was found between the percentage of circulating MDSCs and CD247 expression in the CRC-patients (FIG. 7D), suggesting an impaired immune status associated with the disease. To examine the impact of FOLFOX and FOLFIRI on immune parameters of stage IV CRC patients, the effects of these drugs on the kinetics of MDSC levels and the association with CD247 expression were examined. All patients were treated with chemotherapy for at least six months. PBL analysis revealed decreased levels of circulating MDSCs following FOLFOX treatments (FIG. 7E), which were associated with a tendency of up-regulated CD247 expression (FIG. 7F). By contrast, during the course of FOLFIRI treatment of CRC patients the MDSC percentage continuously increased (FIG. 7G), correlating with CD247 down-regulation (FIG. 7H). These results suggest an adverse impact of the different chemotherapies on the CRC-patients' immune status.

CPT11 but not 5-Fluoruracil (5FU) Treatment Increases MDSC Accumulation at the Tumor Site and Support CRC Growth

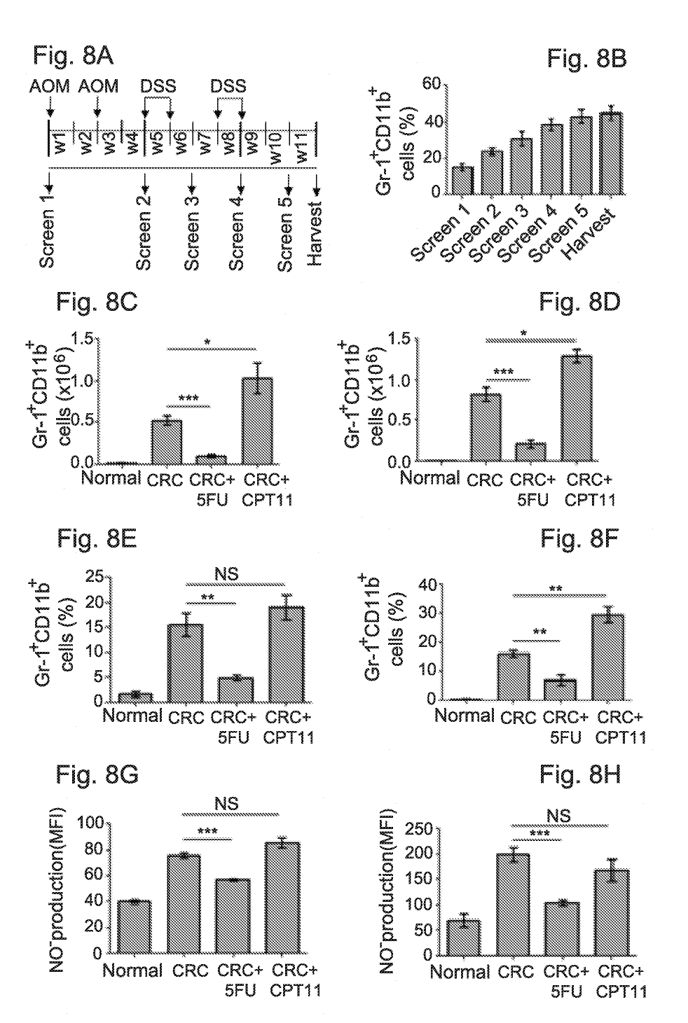

To further investigate whether 5FU and CPT11 display an adverse effect on the immune system, a mouse inducible CRC model based on AOM-DSS treatments was used (FIG. 8A). In this model, the development of CRC is accompanied by chronic inflammation and immune-suppression, reminiscent of spontaneous human CRC (18). Kinetic analysis of MDSC (defined as Gr-1+CD11b⁺ cells) during CRC development revealed their gradual accumulation in the blood along with disease progression (FIG. 8B). When MDSC level became stable and adenomas were evident, mice were treated with either CPT11 or 5FU for three weeks. Histopathology of colonic late neoplasms developed in the AOM/DSS-treated mice was determined by hematoxylin-eosin stain. This analysis revealed that CPT11 monotherapy did not prevent tumorigenesis as no apparent differences in tumor loads within the colon were observed when compared to untreated CRC mice. This stood in sharp contrast to the dramatic effect of 5FU towards a decreased tumor load and recovery of colon architecture.

Moreover, 5FU and CPT11 were found to display opposite effects on β-catenin localization in CRC colons. Colons isolated from the normal and the untreated CRC-mice untreated or CRC-mice treated with 5FU or CPT11 were subjected to immunohistological staining with anti-β-catenin antibodies, as described in the methods. The immunohistochemical analysis showed a massive β-catenin accumulation in the nuclei of tumor cells both in colons from untreated and CPT11 treated CRC-mice, suggesting tumor progression (15).

Histological analyses of colons from CPT11 treated CRC-mice demonstrated not only a loss of entire crypts and surface epithelial layer, but also a massive leukocyte infiltration into the mucosa. Importantly, immunohistochemical probing of MDSCs within the colon revealed elevated levels of the cells in untreated and in CPT11 treated but not in 5FU treated CRC-mice. The same correlation between MDSC accumulation and the given treatment was also observed when testing tumors in colons. Analysis of cells generated from the colon lamina propria (LP) (FIG. 8C) and epithelium (FIG. 8D), depicted increased MDSC numbers in the CPT11 treated as compared to untreated CRC-mice. A significantly reduced MDSC infiltration was observed in both the LP (FIG. 8E) and epithelium (FIG. 8F) following 5FU treatment. Furthermore, a significant reduction of NO⁻ production was found in LP (FIG. 8G) and epithelial (FIG. 8H) MDSCs from 5FU treated CRC-mice, whereas after CPT11 treatment the NO⁻ levels remained elevated as compared to the CRC untreated mice. These results strengthen the CPT11 harmful effects supporting MDSC accumulation and suppressive features at the tumor site.

CPT11 Treatment Increases Systemic Immunosuppression and Counteracts 5FU Beneficial Effects

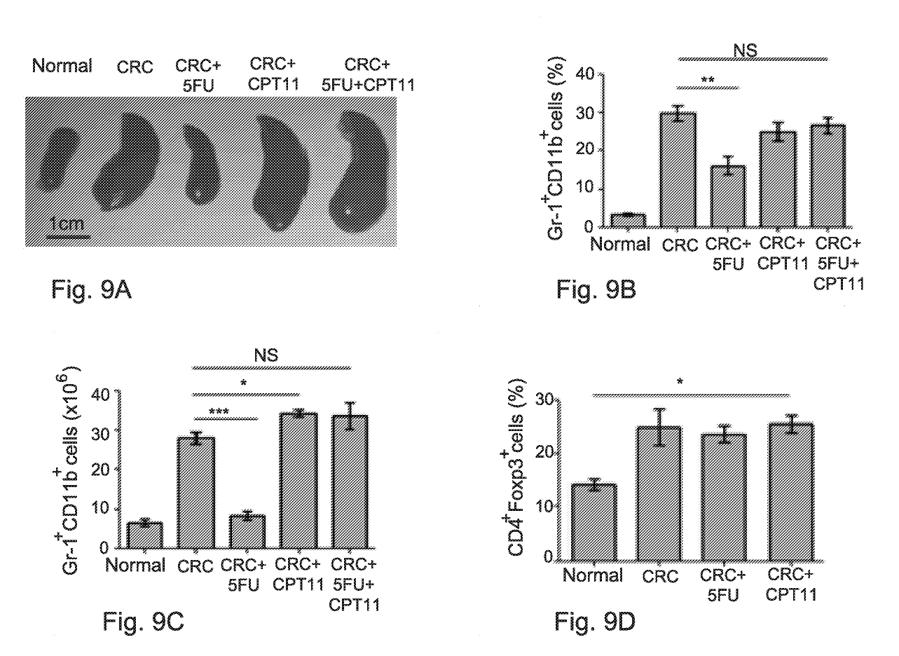

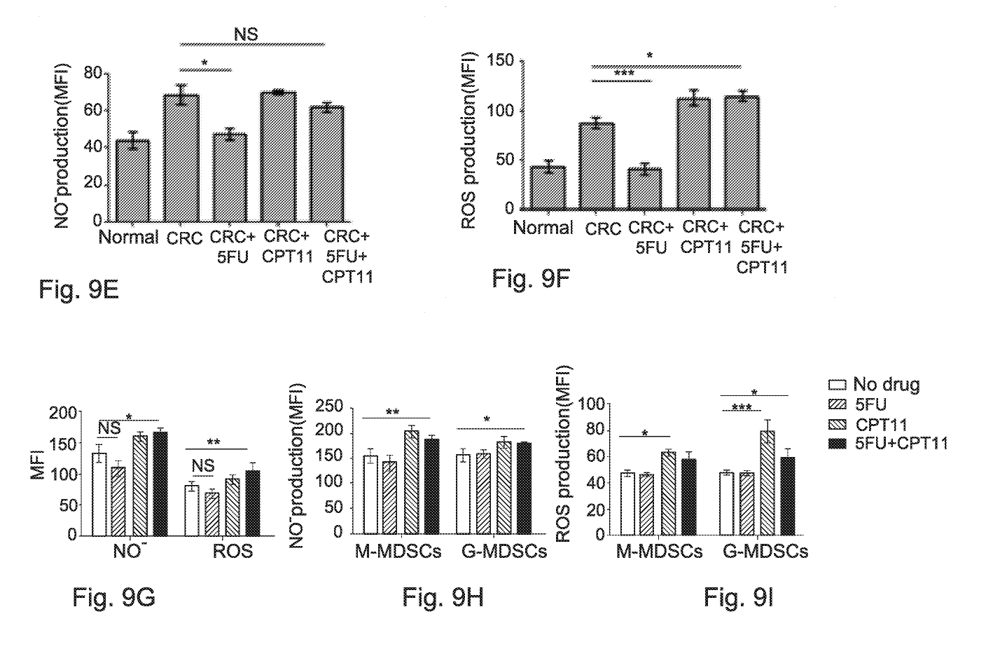

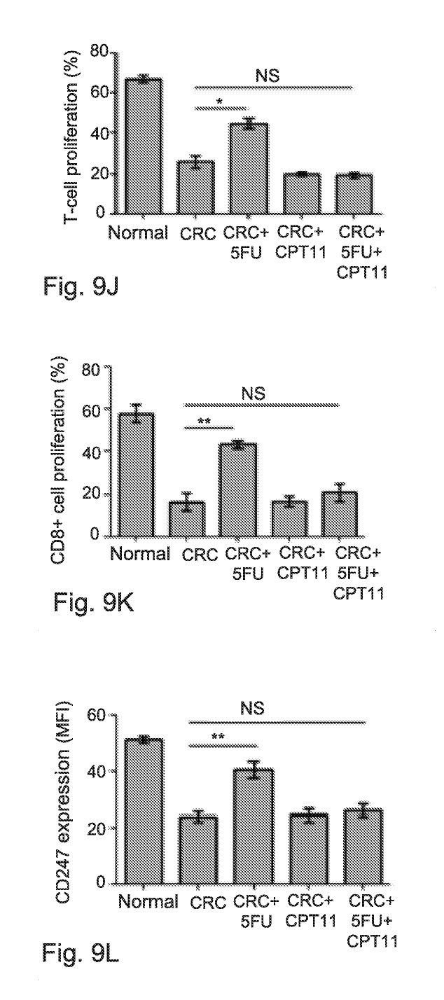

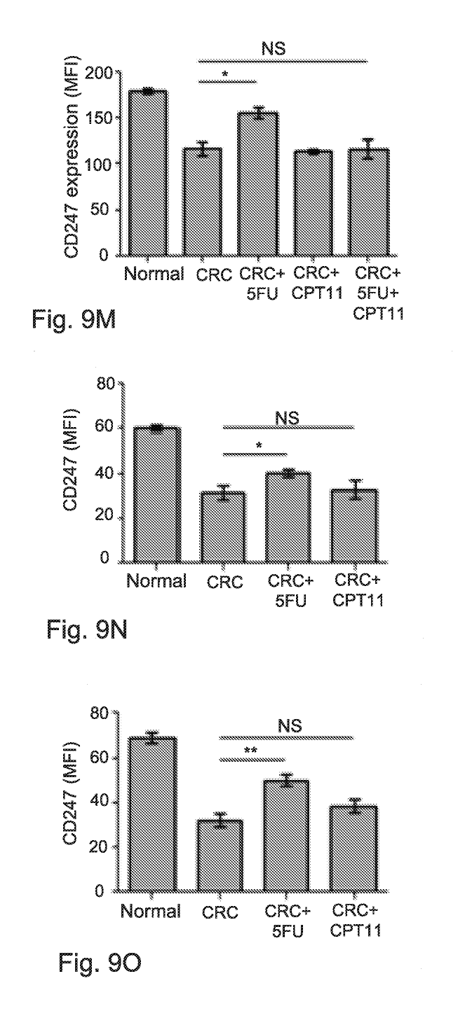

In this experiment, the effects of CPT11 and 5FU on the systemic immunosuppressive state were examined. As shown by the following experiment, a combined 5FU/CPT11 therapy abrogates recovery from immunosuppression during CRC progression. CRC-mice were either subjected to 5FU, CPT11 or a 5FU/CPT11 combination starting at week eight, or were left untreated. Three weeks after the second DSS treatment mice were sacrificed and spleens were analyzed. Untreated, CPT11- and 5FU/CPT11-treated CRC-mice display stronger inflammatory response as indicated by the enlarged spleen size as compared to 5FU treated CRC- or control-mice (FIG. 9A), and by the significantly decreased MDSC numbers in spleens of 5FU treated CRC-mice as compared to those of the untreated CRC-mice or those treated with CPT11 or 5FU/CPT11 (FIGS. 9B and 9C). Interestingly, none of the monotherapies altered the high percentage of regulatory T-cells (CD4⁺Foxp3⁺ Tregs) observed in CRC-mice (FIG. 9D), showing that mainly MDSCs are affected by 5FU and CPT11.

Moreover, while MDSCs from 5FU treated CRC-mice displayed a significantly reduced NO⁻ and ROS production, MDSCs from CPT11 or 5FU/CPT11 treated CRC-mice displayed elevated levels, as compared to untreated CRC-mice (FIGS. 9E and 9F). MDSCs isolated from spleens of CRC-mice (n=5) were cultured ex vivo in the presence of 2.5 μmol/L of 5FU, CPT11 or 5FU and CPT11 for 24 h. The Ex vivo experiments showed that 5FU administration did not alter NO⁻ or ROS production in purified cultured MDSCs, but the addition of CPT11 or 5FU+CPT11 to the medium resulted in their elevation (FIG. 9G), suggesting a direct effect of the drugs on MDSC suppressive features. Both monocytic (CD11b⁺ Ly6C^{high} Ly6G⁻) and granulocyte (CD11b Ly6C^{low} Ly6G⁺) cell populations showed increased NO⁻ (FIG. 9H) and ROS (FIG. 9I) production upon CPT11 or 5FU+CPT11 treatment. However, the monocytic population displayed a more pronounced NO⁻ production, while granulocytic population showed more ROS production (FIGS. 9G and 9H). Thus, CPT11 supports the immunosuppressive environment when applied alone or in a combination with 5FU, affecting the whole MDSC population.

Next, the effect of the given chemotherapies on the immune status was evaluated. This was performed by testing the function of the whole T-cell population (FIG. 9J) as well as the CD8⁺ T-cells (FIG. 9K) in all experimental groups along with measurements of the expression levels of CD247 in CD3⁺ (FIG. 9L) and in CD8⁺ T-cells (FIG. 9M). T-cell proliferative response was assessed by monitoring cell divisions of gated CFSE-labeled Thy1.2⁺ (CD90⁺) T-cells upon TCR-CD28 mediated activation. The results revealed a decreased T-cell proliferative ability following both CPT11 and 5FU/CPT11 treatments. In contrast, 5FU treatment of CRC-mice did not affect T-cell proliferation, as compared to untreated CRC-mice. Moreover, T-cell function was correlated with CD247 expression levels; low CD247 levels were obtained in T-cells from CRC-mice untreated and treated with CPT11 or a 5FU/CPT11 combination, while elevated levels were obtained upon 5FU treatment. Similar results were found in T-cells isolated from the LP and epithelium, inversely correlating with the local generated immunosuppressive environment (FIGS. 9N and 9O). Thus, CPT11 and 5FU adversely affect not only the tumor but also the immune system, pointing at the dominating harmful effects of CPT11 when combined with 5FU.

CPT11 Harmful Effects on the Host's Immune Function are Mediated Via MDSCs

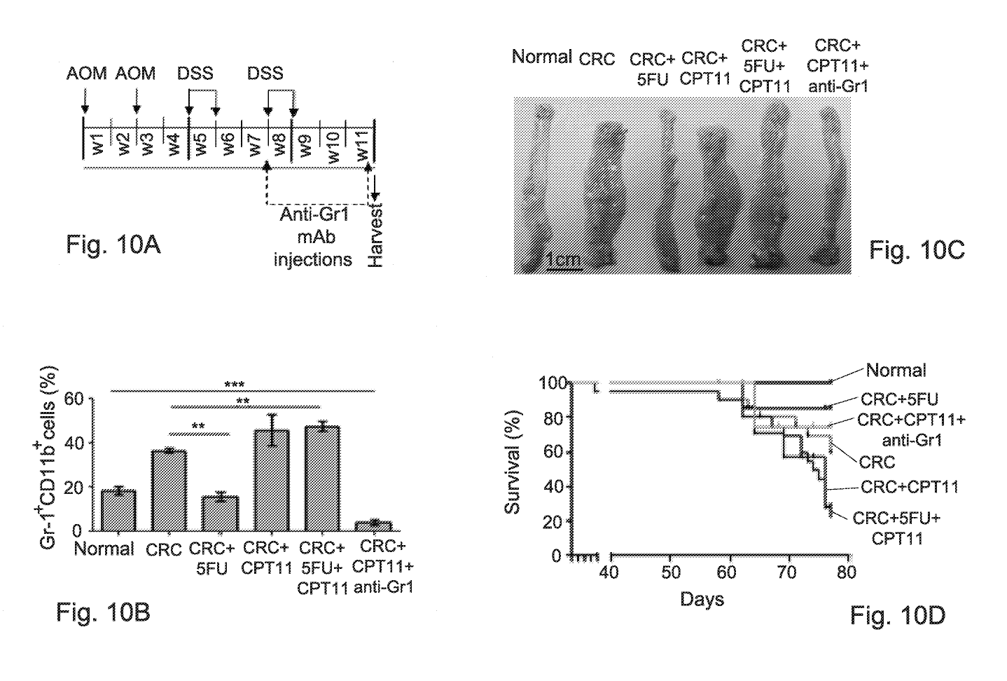

The observed MDSC elevation and increased tumor load in CPT11 treated CRC-mice as described above, suggests an impact of CPT11 on MDSC-induced cancer progression. Therefore, MDSC depletion could reduce the harmful effect of CPT11 on the immune status, and thereby enhance the anti-tumor effect. CRC-mice were subjected to either 5FU, CPT11 or a 5FU/CPT11 combination starting at week eight, or were untreated. At the same day of the second DSS administration, CPT11-treated CRC-mice were randomly separated into two groups; one group continued with CPT11 treatment while the second group was treated with anti-Gr1 mAb for MDSC depletion in addition to CPT11 treatment. A schematic description of the CRC mouse model in which the MDSCs were depleted by Gr1 mAb administration is shown in FIG. 10A. Three weeks after the second DSS treatment mice were sacrificed and PBLs (FIG. 10B) and colons (FIG. 10C) were analyzed. Indeed, in vivo MDSC depletion in the CPT11 treated CRC-mice, as indicated by the negligible MDSC levels as compared to CPT11 and 5FU/CPT11 treated CRC-mice (FIG. 10B), led to an almost complete regression of the tumors (FIG. 10C). Moreover, histopathological analysis using hematoxylin-eosin staining of colon sections revealed a differentiated adenocarcinoma in the colons of CRC-mice, CPT11 and 5FU/CPT11 treated CRC-mice, while only few aberrant crypt foci and tumors were detected in colons of 5FU treated CRC-mice and CPT11 treated MDSC depleted CRC-mice. This pattern indicates a beneficial effect of 5FU that attenuates CRC progression with a harmful contribution of CPT11, supporting immunosuppression and tumor progression via its effects on MDSCs. The daily recorded vitality and survival of the mice show a rapid deterioration, with increased death rates in CRC-mice treated with CPT11 or 5FU/CPT11, as compared to the 5FU, or CPT11 treated MDSC depleted CRC-mice, or even to untreated CRC-mice (FIG. 10D).

MDSCs are Insensitive to Apoptosis Under CPT11 Treatment but Become Susceptible after 5FU Treatment

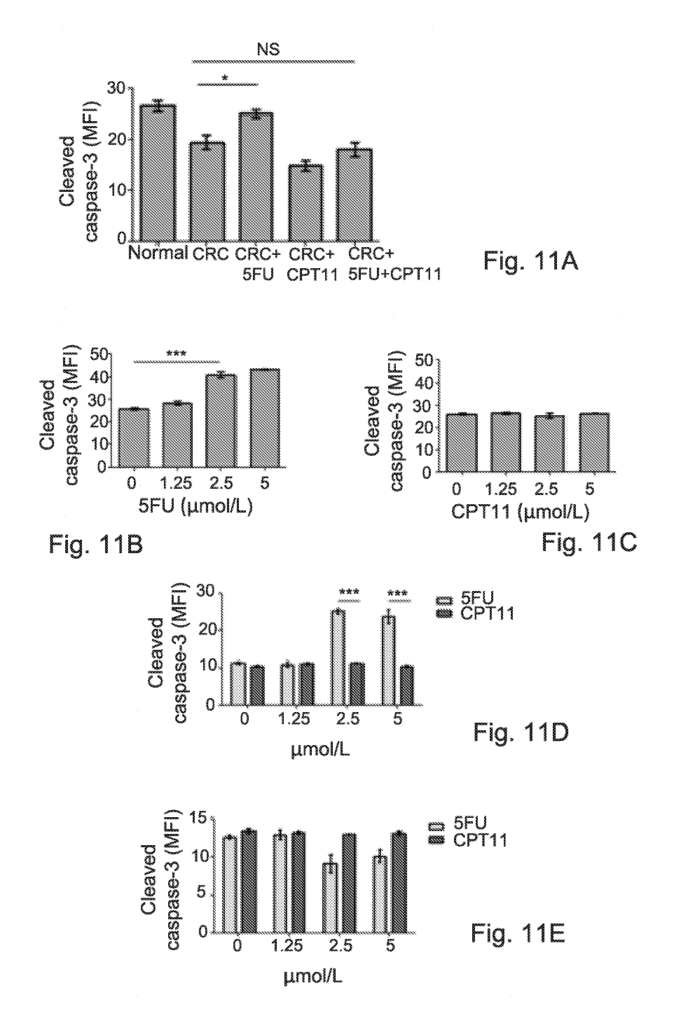

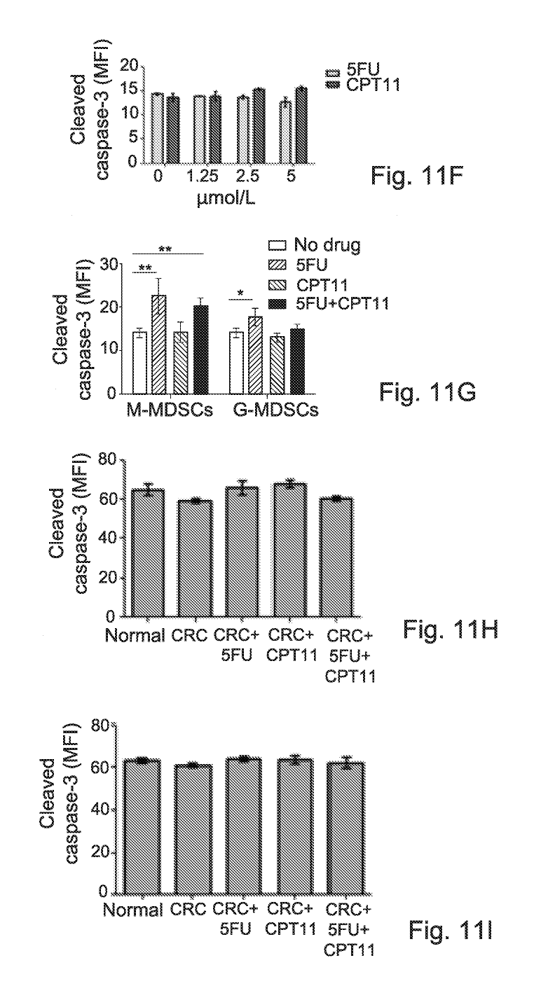

Next, the mechanisms responsible for the opposite effects of 5FU and CPT11 on MDSC accumulation were investigated. One possible explanation for the observed differential effect of these drugs on MDSC levels could be a change in the sensitivity of the MDSCs to apoptosis, as previously reported for 5FU (11). Therefore, Splenic MDSCs from each group were analyzed for the expression of activated (cleaved) caspase-3 by flow cytometry analysis. To assess the direct effect of 5FU and CPT11 on cleaved caspase-3 expression, primary MDSCs isolated from spleens of CRC mice (n=6) were ex-vivo incubated with various doses of the drugs for 3 days and subjected to flow cytometry analysis. To assess which cells are affected by the chemotherapeutic drugs, MDSCs isolated from spleens of CRC mice were cultured ex-vivo with 10 ng/ml of GM-CSF in the absence or presence of scaled-doses (0, 1.25, 2.5, and 5 mol/L) of 5FU or CPT11 for 3 days. Cleaved caspase-3 levels were then evaluated on the primary MDSCs, differentiated CD11c⁺CD11b⁺DCs and F4/80⁺CD11b⁺ macrophages. Indeed, a significant increased cleaved caspase-3 expression, an indicator for apoptosis, was observed within spleen MDSCs from 5FU treated CRC-mice, similar to that detected in MDSCs from control-mice (FIG. 11A). In contrast, following CPT11 or 5FU/CPT11 treatment, MDSCs displayed decreased cleaved caspase-3 levels, as in spleen MDSCs of untreated CRC-mice (FIG. 11A). Moreover, ex vivo studies revealed that 5FU but not CPT11 leads to cleaved caspase-3 up-regulation in purified cultured MDSCs in a dose-dependent manner (FIGS. 11B and C). Interestingly, 5FU-induced apoptosis was evident only in non-differentiated MDSCs (FIG. 11D) while DCs (FIG. 11E) and macrophages (FIG. 11F) were insensitive, suggesting an exclusive effect of 5FU on the immature myeloid cell population.

In addition, 5FU and CPT11 were found to differently affect monocytic/granulocytic MDSC sensitivity to apoptosis. MDSCs isolated from spleens of CRC-mice (n=5) were cultured ex vivo in the presence of 2.5 mol/L of 5FU, CPT11 or 5FU and CPT11 for 3 days. Cultured MDSCs were analyzed for cleaved caspase-3 expression (MFI) by flow cytometry analysis gaiting on CD11b⁺Ly6C^{high} Ly6G monocytic MDSCs (M-MDSCs) and CD11b⁺Ly6C^{low}Ly6G⁺ granulocytic MDSCs (G-MDSCs) sub-populations. It was found that 5FU controls both monocytic and granulocytic purified cultured MDSC populations, with the monocytic population being more sensitive, as indicated by the enhanced cleaved caspase-3 expression upon 5FU addition or when combined with CPT11 (FIG. 11G).

The drugs did not affect the apoptotic state of other immune cells such as T (CD3⁺) lymphocytes or B (B220⁺) lymphocytes (FIGS. 11H and I). These results underscore the direct apoptotic effects of 5FU on immature MDSCs as opposed to the apoptosis insensitivity of MDSCs to the CPT11 or CPT11/5FU treatments.

5FU and CPT11 Directly Affect Myeloid Cell Maturation and Suppressive Activity

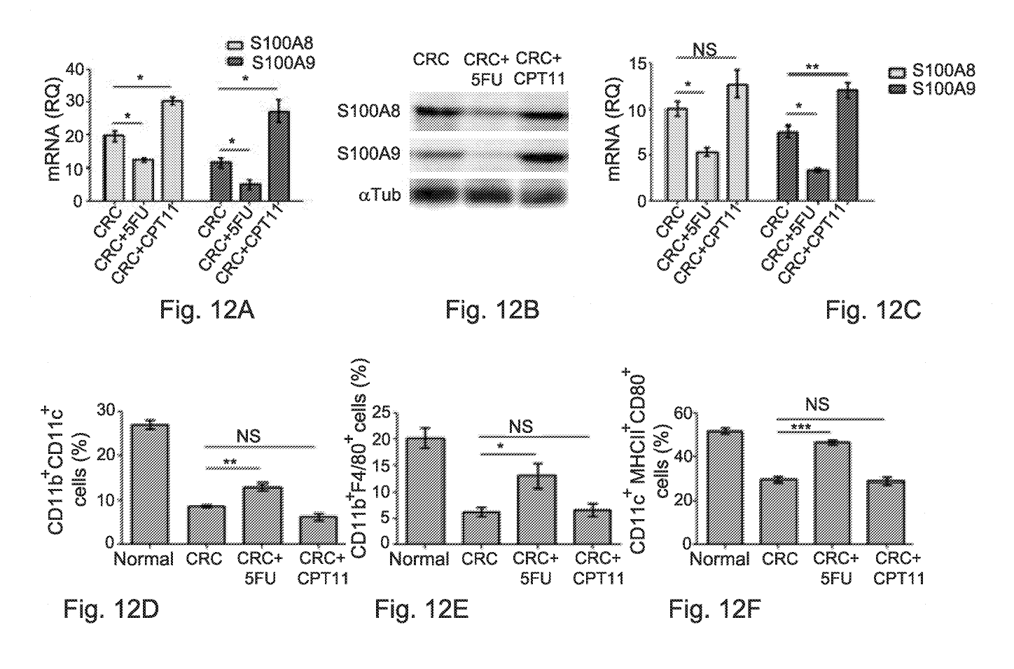

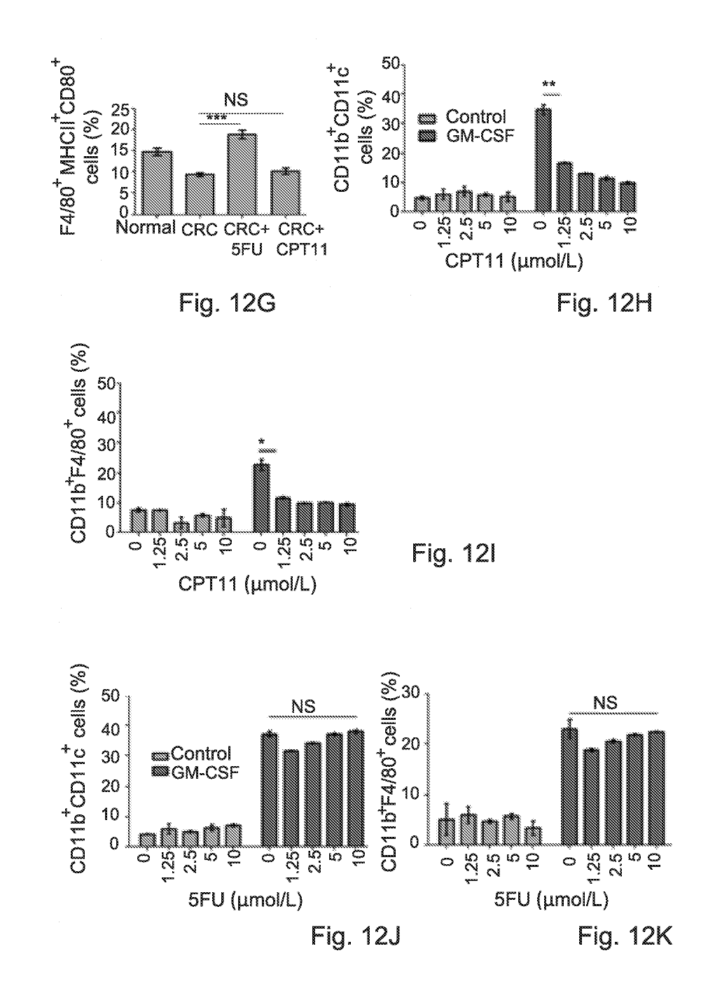

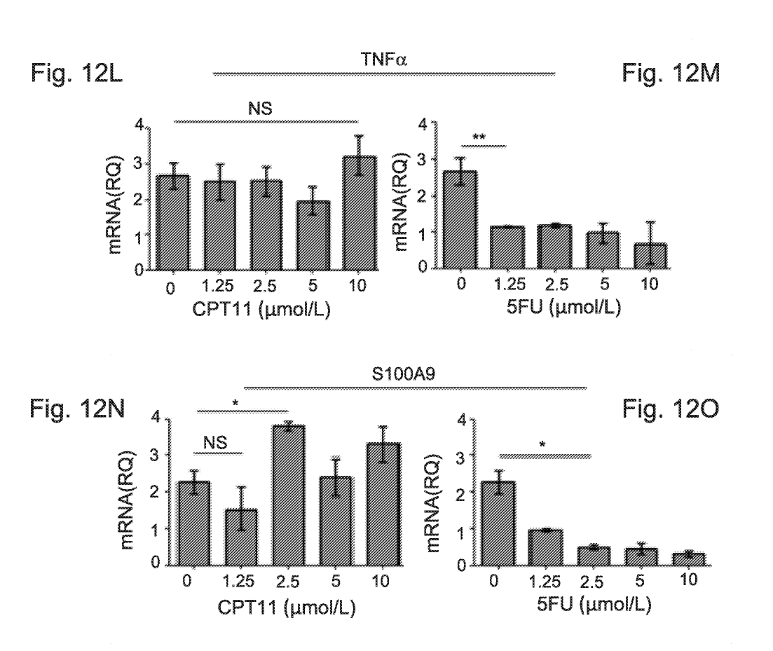

Next, the effect of 5FU and CPT11 on MDSC maturation was examined. For that purpose the expression of the S100A8/9 pro-inflammatory proteins was assessed. The S100A8/9 pro-inflammatory proteins are induced in the course of tumorigenesis and chronic inflammation and play a role in controlling MDSC accumulation and retention in their immature suppressive state (7, 16, 19). S100A8/9 mRNA and protein levels were evaluated in MDSCs isolated from the spleen of CRC-mice, or CRC-mice treated with 5FU or CPT11 (n=4), α-Tubulin levels served as a control. While 5FU treatment of CRC-mice induced a significant decrease in S100A8/9 mRNA and protein levels in the spleen as compared to control-mice, increased S100A8/9 levels were observed following CPT11 treatment (FIGS. 12A and B). Similar results were obtained when assessing the colon (FIG. 12C), suggesting that 5FU supports MDSC transition from an immature suppressive stage towards differentiated non-suppressive myeloid phenotype (16). Indeed, 5FU treatment of CRC-mice resulted in a significant shift towards differentiated DCs and macrophages (FIGS. 12D and 12E) and to matured antigen presenting cells (APCs), shown by the induced CD80 and MHCII expression (FIGS. 12F and 12G). In contrast, CPT11 treatment blocked myeloid cell differentiation in vivo (FIGS. 12D-12G).

When testing the ex vivo direct effects of the drugs on GM-CSF mediated CRC-derived MDSC differentiation, it was found that, similar to the in vivo effects, CPT11 prevented cell differentiation after both 48 h (data not shown) and 72 h (FIGS. 12H and 12I) as compared to MDSCs incubated with GM-CSF only. In contrast, 5FU enabled MDSC differentiation to DCs and macrophages (FIGS. 12J and 12K). Interestingly, ex vivo CPT11-mediated MDSC differentiation blockade was associated with increased mRNA levels of the pro-inflammatory mediators TNFα (FIGS. 12L and 12M) S100A9 (FIGS. 12N and 12O), while the 5FU-induced MDSC differentiation correlated with decreased levels of these factors. Thus, 5FU directly affects the differentiation pathway of MDSCs whereas CPT11 exhibits a differentiation blockade capacity when added to GM-CSF-treated MDSCs.

5FU and CPT11 Opposing Effects on MDSCs are Tumor Independent

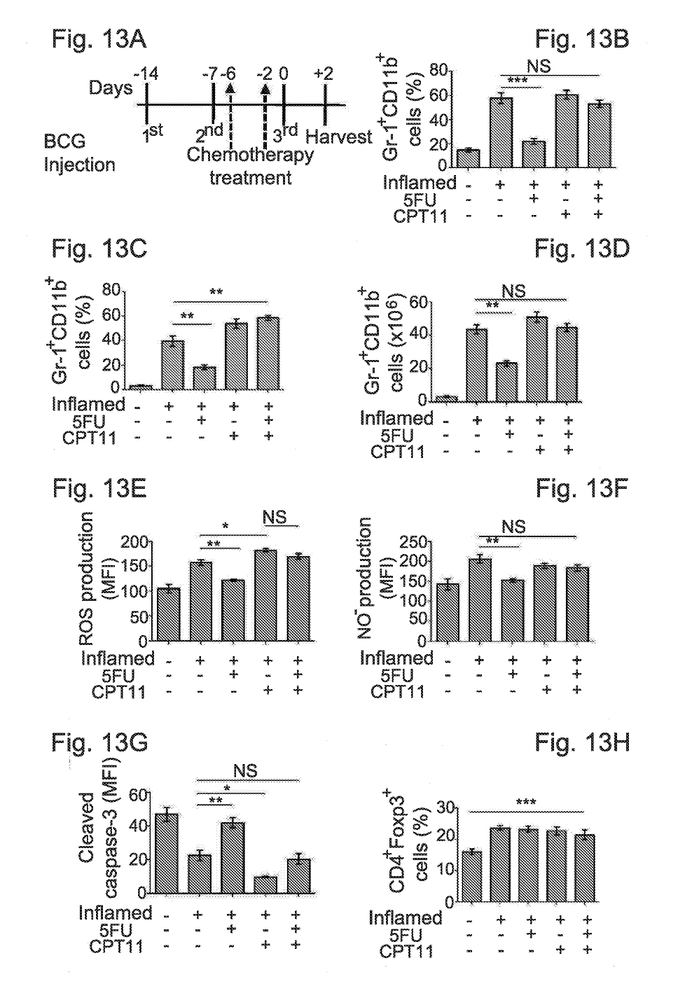

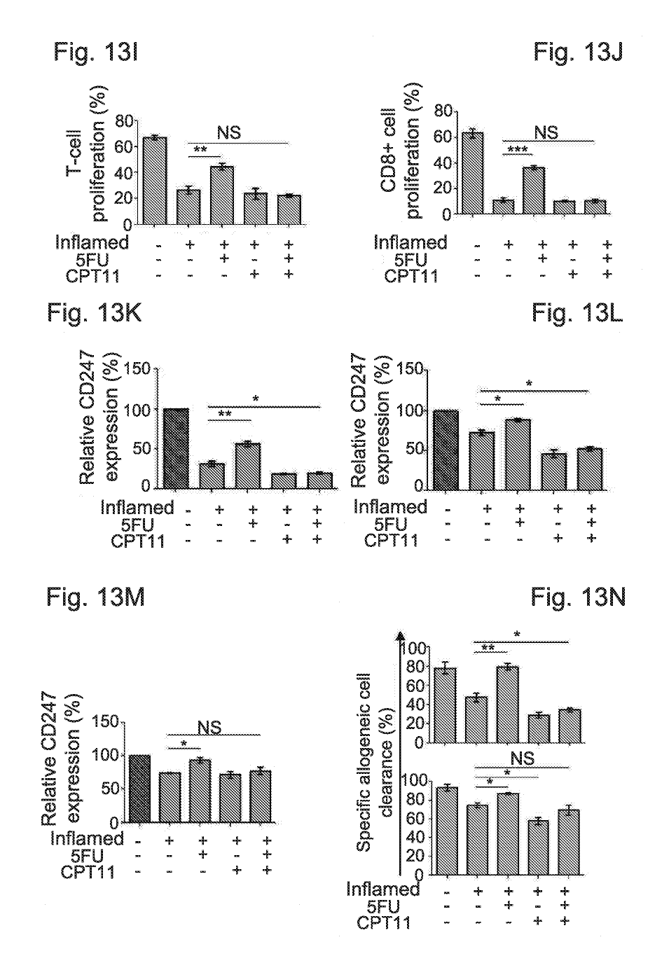

Next, the immunoregulatory effects of 5FU and CPT11 were examined in a mouse model for chronic inflammation and associated immunosuppression (10), as described in the materials and method section above and in FIG. 13A. This experiment was performed in order to investigate whether the differential effects of the drugs are related to the presence of the tumor. Briefly, the mouse model for chronic inflammation was established by three repeated injections of heat-killed BCG bacteria. A day after the second BCG injection, mice were treated twice a week with 5FU, CPT11 or a 5FU/CPT11 combination. The 5FU beneficial and CPT11 harmful effects were similar to those observed in the CRC model. 5FU significantly reduced MDSC levels (FIGS. 13B-13D) and ROS and NO⁻ production (FIGS. 13E and 13F). Treatment with 5FU also elevated cleaved caspase-3 levels (FIG. 13G), as compared to inflamed-untreated mice. In contrast, CPT11 or 5FU/CPT11 treatments induced opposite effects (FIGS. 13 B-G). No changes in CD4⁺Foxp3⁺ Tregs percentage were detected (FIG. 13H), confirming that these chemotherapies specifically affect MDSCs.

Next, it was assessed whether the 5FU and CPT11 opposite effects on MDSCs have different impacts on the host's immune competence. Assessment of the drugs' effects on total T-cell activity, and specifically CD8⁺ T-cells revealed a significant recovery of CD247 expression in the spleen and PBLs as well as T-cell proliferation following 5FU but not CPT11 or 5FU/CPT11 treatments (FIGS. 13I-13L). Similar effects were also detected when analyzing NK-cells; down-regulated CD247 expression detected in NK-cells from chronically inflamed mice was almost completely recovered upon 5FU but not CPT11 treatment (FIG. 13M). Since NK-cell activity is mediated via natural cytotoxicity receptors (NCRs), which associate with and depend on CD247, NK-cell in vivo function was assessed by monitoring the clearance of adoptively transferred allogeneic cells. A complete recovery of NK-cell activity both in the spleen and PBLs was detected following 5FU treatment (FIG. 13N), along with a significant decreased clearance of allogeneic cells after CPT11 or 5FU/CPT11 treatments. These results indicate that 5FU and CPT11 opposite effects on MDSCs are tumor-independent, displaying a significant impact on effector T- and NK-cell responsiveness under chronic inflammatory conditions.

### REFERENCES

1. Terzic J, et al. Inflammation and colon cancer. Gastroenterology 2010; 138: 2101-14 e5.
2. Zhang B, et al. Circulating and tumor-infiltrating myeloid-derived suppressor cells in patients with colorectal carcinoma. PLoS One 2013; 8: e57114.
3. Sun H L, et al. Increased frequency and clinical significance of myeloid-derived suppressor cells in human colorectal carcinoma. World J Gastroenterol 2012; 18: 3303-9.
4. Coussens L M and Werb Z. Inflammation and cancer. Nature 2002; 420: 860-7.
5. Bruchard M, et al. Chemotherapy-triggered cathepsin B release in myeloid-derived suppressor cells activates the Nlrp3 inflammasome and promotes tumor growth. Nat Med 2013; 19: 57-64.
6. Bunt S K, et al. Inflammation enhances myeloid-derived suppressor cell cross-talk by signaling through Toll-like receptor 4. J Leukoc Biol 2009; 85: 996-1004.
7. Cheng P, et al. Inhibition of dendritic cell differentiation and accumulation of myeloid-derived suppressor cells in cancer is regulated by S100A9 protein. J Exp Med 2008; 205: 2235-49.
8. Kanterman J, et al. New insights into chronic inflammation-induced immunosuppression. Semin Cancer Biol 2012; 22: 307-18.
9. Ramakrishnan R, and Gabrilovich D I. Mechanism of synergistic effect of chemotherapy and immunotherapy of cancer. Cancer Immunol Immunother 2011; 60: 419-23.
10. Vaknin I, et al. A common pathway mediated through Toll-like receptors leads to T- and natural killer-cell immunosuppression. Blood 2008; 111: 1437-47.
11. Vincent J, et al. 5-Fluorouracil selectively kills tumor-associated myeloid-derived suppressor cells resulting in enhanced T-cell-dependent antitumor immunity. Cancer Res 2010; 70: 3052-61.
12. Hillner B E, et al. Cost-effectiveness projections of oxaliplatin and infusional fluorouracil versus irinotecan and bolus fluorouracil in first-line therapy for metastatic colorectal carcinoma. Cancer 2005; 104: 1871-84.
13. Nelson M A, et al. A comparison of mortality and costs associated with FOLFOX versus FOLFIRI in stage I V colorectal cancer. J Med Econ 2011; 14: 179-86.
14. Tournigand C, et al. FOLFIRI followed by FOLFOX6 or the reverse sequence in advanced colorectal cancer: a randomized GERCOR study. J Clin Oncol 2004; 22: 229-37.
15. Popivanova B K, et al. Blocking TNF-alpha in mice reduces colorectal carcinogenesis associated with chronic colitis. J Clin Invest 2008; 118: 560-70.
16. Sade-Feldman M, et al. Tumor necrosis factor-alpha blocks differentiation and enhances suppressive activity of immature myeloid cells during chronic inflammation. Immunity 2013; 38: 541-54.
17. Drakes M L, et al. Isolation and purification of colon lamina propria dendritic cells from mice with colitis. Cytotechnology 2004; 46: 151-61.
18. De Robertis M, et al. The AOM/DSS murine model for the study of colon carcinogenesis: From pathways to diagnosis and therapy studies. J Carcinog 2011; 10: 9.
19. Sinha P, et al. Proinflammatory S100 proteins regulate the accumulation of myeloid-derived suppressor cells. J Immunol 2008; 181: 4666-75.

1-27. (canceled)

28. A method for predicting a cancer patient's response to treatment with an immunotherapeutic agent, said method comprising the steps of:
(a) measuring the amount of myeloid derived suppressor cells (MDSC) having the profile CD11b+CD33+HLA-DR− and/or CD11b+CD33+HLA-DRlow, in at least one biological sample being a whole blood sample obtained from the patient, wherein said whole blood sample is a fresh sample, a frozen sample, a preserved sample or a cryopreserved sample;
(b) determining whether the amount of MDSC of said profile is higher than a predetermined standard value or higher than a control sample; wherein detection of a high amount of MDSC of the above profile in the at least one biological sample as compared with the predetermined standard value or the control sample indicates that the patient will not respond or will poorly respond to treatment with said agent; and
(c) if the patient was not found to be irresponsive or poorly responsive to treatment with said agent, at least one of the following steps are performed:
i) including said patient in a clinical study;
ii) starting or continuing treatment of said patient with said immunotherapeutic agent; or
if the patient was found to be irresponsive or poorly responsive to treatment with said agent, at least one of the following steps are performed:
iii) excluding said patient from a clinical study; and
iv) ceasing treatment of said patient with said immunotherapeutic agent.

29. A method according to claim 28, wherein said method is for at least one of the following:
(a) including or excluding patients in a clinical study;
(b) deciding whether the patient should start, continue or cease therapy with said immunotherapeutic agent; or
(c) deciding which combination of immunotherapeutic agents should be used.

30. A method for monitoring a cancer patient's response to treatment with an immunotherapeutic agent, said method comprising the steps of:
(a) measuring the amount of myeloid derived suppressor cells (MDSC) having the profile CD11b+CD33+HLA-DR− and/or CD11b+CD33+HLA-DRlow, in at least one biological sample being a whole blood sample obtained from the patient, wherein said whole blood sample is a fresh sample, a frozen sample, a preserved sample or a cryopreserved sample;
(b) determining whether the amount of MDSC of said profile is higher than a predetermined standard value or higher than a control sample; wherein detection of a high amount of MDSC of the above profile in the at least one biological sample as compared with the predetermined standard value or the control sample indicates that the patient is not responding or is poorly responding to treatment; and
(c) if the patient was not found to be irresponsive or poorly responsive to treatment with said agent, continuing treatment of said patient with said immunotherapeutic agent; or
if the patient was found to be irresponsive or poorly responsive to treatment with said agent, at least one of the following steps are performed:
i) discontinuing treatment with said immunotherapeutic agent; or
ii) combining the treatment with said immunotherapeutic agent with at least one additional compound being an anti-inflammatory and/or an anti-MDSC therapeutic agent.

31. A method according to claim 30, wherein said method is used for determining the patient's therapeutic regime by at least one of the following:
(a) discontinuing treatment with said immunotherapeutic agent; or
(b) combining the treatment with said immunotherapeutic agent with at least one additional compound being an anti-inflammatory and/or an anti-MDSC therapeutic agent.

32. The method of claim 28, wherein said measuring of step (a) is performed prior to treatment with said immunotherapeutic agent.

33. The method of claim 30, wherein said measuring of step (a) is performed at least once, preferably more than once, during treatment with said immunotherapeutic agent.

34. A method according to claim 28, wherein said immunotherapeutic agent is selected from the group consisting of therapeutic antibodies, immune checkpoints inhibitors, cytokines, tumor infiltrating lymphocytes (TIL) and cancer vaccines.

35. A method according to claim 28, wherein the cancer is selected from the group consisting of adrenal cancer, bladder cancer, brain cancer, breast cancer, cervical cancer, colorectal carcinoma, endometrial cancer, gastro-intestinal cancers, head and neck squamous cell carcinoma, leukemia, malignant lymphoma, including Hodgkin's lymphoma, liver cancer, melanoma, ovarian cancer, pancreatic cancer, prostate cancer, renal cancer, sarcoma, small cell lung cancer, non-small cell lung cancer, and thyroid cancer.

36. A method according to claim 35, wherein the cancer is melanoma or colorectal carcinoma.

37. The method of claim 28, wherein said measuring of step (a) is performed by a method comprising the step of contacting detecting molecules specific for MDSC with the biological sample.

38. The method of claim 37, wherein said detecting molecules are labeled detecting molecules.

39. The method of claim 37, wherein said detecting molecules are attached to a substrate.

40. The method of claim 37, wherein said detecting molecules are antibodies that specifically recognize and bind MDSC having the profile CD11b⁺CD33⁺HLA-DR⁻ and/or CD11b⁺CD33⁺HLA-DR^{low}.

41. The method of claim 28, wherein said method further comprises after step (b) the step of at least one of:
(A) determining the expression levels of S100A8 and/or S100A9 proteins or encoding mRNA in said biological sample;
(B) determining the levels of cleaved caspase 3 in said biological sample;
(C) determining at least one of the level and/or activity of arginase 1 and iNOS in said biological sample;
(D) determining at least one of intracellular nitric oxide (NO) and reactive oxygen species (ROS) in said biological sample;
(E) determining the level of lactate dehydrogynase (LDH) in said biological sample; and
(F) determining the level of MDSC suppressive activity on T cells in said biological sample, wherein
determining elevated levels of NO, elevated levels of ROS, elevated levels of S100A8 and/or S100A9 proteins, low levels of cleaved caspase 3, and MDSC suppressive activity on T cells indicates that the patient will not respond to treatment with said immunotherapeutic agent, or that the therapeutic regimen of said patient should be altered.

42. A method of treating a cancer patient with an immunotherapeutic agent, said method comprising the steps of:
(a) determining whether said patient is responsive to the immunotherapeutic agent in accordance with the method of claim 28; and
(b) wherein the patient was determined to be responsive, administering to the patient an effective amount of said immunotherapeutic agent.

43. A method for selecting a melanoma patient suitable for receiving treatment with an immunotherapeutic agent, said method comprising the steps of:
(a) measuring the amount of myeloid derived suppressor cells (MDSC) having the profile CD11b+CD33+HLA-DR− and/or CD11b+CD33+HLA-DRlow, in at least one biological sample being a whole blood sample obtained from the patient, wherein said whole blood sample is a fresh sample, a frozen sample, a preserved sample or a cryopreserved sample; and
(b) determining whether the amount of MDSC is lower than a predetermined standard value or lower than a control sample;
wherein
(i) said therapeutic agent is Ipilimumab or lambrolizumab or a combination thereof, and wherein
(ii) detection of a low amount of MDSC in the at least one biological sample as compared with the predetermined standard value or the control sample, indicates that the patient is suitable for receiving treatment with Ipilimumab or lambrolizumab or a combination thereof; and
(c) treating said patient with Ipilimumab or lambrolizumab or a combination thereof if the patient was found to be suitable for receiving said treatment.

44. A kit comprising:
(a) detecting molecules specific for MDSC having the profile CD11b+CD33+HLA-DR− and/or CD11b+CD33+HLA-DRlow; and optionally further comprising at least one of the following:
i) at least one detecting molecule for determining LDH levels;
ii) at least one detecting molecule specific for at least one of S100A8, S100A9, cleaved caspase 3, iNOS, arginase 1, NO, ROS, CD247 or SNX9;
iii) secondary agents and/or buffers for performing detection of MDSC, LDH and at least one of S100A8, S100A9, cleaved caspase 3, iNOS, arginase 1, NO or ROS; and
(b) instructions for use comprising instructions for carrying out the detection in at least one biological sample being a whole blood sample, wherein said whole blood sample is a fresh sample, a frozen sample, a preserved sample or a cryopreserved sample,
wherein said kit is for use in a method for predicting a cancer patient's response to treatment with an immunotherapeutic agent, or for use in a method for determining whether the therapeutic regimen of a cancer patient should be altered.

45. The kit of claim 44, wherein said detecting molecules are labeled detecting molecules.

46. The kit of claim 44, wherein said detecting molecules are attached to a substrate.

47. The kit of claim 46, wherein said detecting molecules comprise antibodies that specifically recognize and bind MDSC having the profile CD11b⁺CD33⁺HLA-DR⁻ and/or CD11b⁺CD33⁺HLA-DR^{low}.


SEQUENCE LISTING (8 sequences)

SEQ ID NO: 1 - DNA, 23 bp, Mus musculus
ggaaatcaccatgccctctacaa

SEQ ID NO: 2 - DNA, 23 bp, Mus musculus
atgccacacccacttttatcacc

SEQ ID NO: 3 - DNA, 22 bp, Mus musculus
ggagcgcagcataaccaccatc

SEQ ID NO: 4 - DNA, 24 bp, Mus musculus
gccatcagcatcatacactcctca

SEQ ID NO: 5 - DNA, 21 bp, Mus musculus
gttaagcagtacagccccaaa

SEQ ID NO: 6 - DNA, 23 bp, Mus musculus
agggcatatccaacaacaaactt

SEQ ID NO: 7 - DNA, 22 bp, Mus musculus
gccaccacgctcttctgtctac

SEQ ID NO: 8 - DNA, 22 bp, Mus musculus
gggtctgggccatagaactgat